(19) United States
(12) Patent Application Publication
(10) Pub. No.: US 2025/0263447 A1
(43) Pub. Date: Aug. 21, 2025

(54) OMPA MUTATIONS ENHANCE OMV PRODUCTION IN BORDETELLA PERTUSSIS

(71) Applicant: INTRAVACC B.V., Bilthoven (NL)

(72) Inventors: Peter André VAN DER LEY, Utrecht (NL); Arjen Jan SLOOTS, Bilthoven (NL); Arnoldus Adrianus Johannes VAN DER ARK, The Hague (NL)

(73) Assignee: Intravacc B.V., Bilthoven (NL)

(21) Appl. No.: 18/043,595

(22) PCT Filed: Sep. 3, 2021
(86) PCT No.: PCT/EP2021/074317

§ 371 Date: Mar. 1, 2023

(30) Foreign Application Priority Data
Sep. 4, 2020 (EP) 20194514.4

(51) Int. Cl. C07K 14/235; A61K 39/00; A61K 39/02

(52) U.S. Cl. CPC C07K 14/235; A61K 39/099; A61K 2039/521

(57) ABSTRACT

The invention pertains to a mutant Bordetella OmpA polypeptide. A Bordetella comprising said mutant polypeptide has a high blebbing phenotype. Hence, the invention further pertains a method for producing OMVs, wherein the method comprises a step of culturing a population of Bordetella bacteria comprising a modified OmpA polypeptide, under conditions conductive for the production of OMVs. In addition, the invention pertains to OMVs produced by a Bordetella comprising a mutant OmpA polypeptide, and the use of such OMVs for the treatment and prevention of a Bordetella infection.

### FIELD OF THE INVENTION

The present invention is in the field of vaccinology and in particular in the field of the prevention or treatment of a Bordetella infection.

The present invention pertains to a modified Bordetella bacterium having an increased OMV production, as well as OMVs obtainable from said modified OMV bacterium. The invention further pertains to compositions comprising the modified Bordetella and/or OMV and the use of said compositions in the prevention and/or treatment of a Bordetella infection.

### BACKGROUND OF THE INVENTION

Pertussis, better known as whooping cough, is a highly infectious respiratory disease caused by the bacterium Bordetella (B.) pertussis. Distinctive for pertussis are severe coughing attacks followed by forced inhalation combined with the characteristic whooping sound. Symptoms may vary by age and/or level of immunization of an individual. Especially infants are at risk to enter a life-threatening situation which can lead to respiratory failure with mortality as result. Besides the life-threatening cases among infants, the disease can cause social and economic impediments in adults. Nowadays pertussis is also more commonly diagnosed in older children and adults instead of newborns and infants.

B. pertussis was first identified in 1906 as the causative agent of pertussis. B. pertussis is a Gram-negative bacterium that specially infects humans. Transmission of the bacteria occurs from human to human by inhalation of respiratory droplets. In most developed countries, a vaccination against the pertussis was implemented between 1940 and 1950. This vaccine consisted of inactivated B. pertussis bacteria in combination with diphtheria and tetanus toxoids. Through the introduction of a vaccination scheme for infants, consisting of three injections during the first half year of life, the incidence of pertussis was significantly reduced. This reduction indicates the effectiveness of these so-called whole-cell pertussis (wP) vaccines. Despite the effectiveness and reduced incidence, wP vaccines were replaced by acellular (aP) vaccines in the late 1990s. The main reason for the replacement was the association of some serious complications from the use of wP vaccines. The aP vaccines contain combinations of some of the most important virulence factors of B. pertussis e.g. pertussis toxin, filamentous haemagglutinin, pertactin and fimbrial proteins 2 and 3. These virulence factors introduce a potent immune response against B. pertussis.

Different strategies for protection of newborns were studied because of the lack of protection of these young infants. First of all the aP vaccination scheme should protect against a pertussis infection, but waning immunity has been described in several studies. In addition, an increase in cases of B. pertussis infections has been reported. Besides the primary vaccination scheme, several other strategies were explored and advised. The cocoon strategy is recommended in the United states by the Centers for Disease Control and prevention (CDC) since 2006. By this strategy, the newborn is aimed to be protected against an infection with B. pertussis by administrating an aP booster vaccine to family members and all close individuals who have regular contact to the newborn. By this strategy, a cocoon is created around the newborn. However, only cocooning is most likely not enough to prevent newborns against pertussis infection. Finances are also a limiting factor in cocooning the newborns, even as additional investments by hospitals. A second strategy studied is the vaccination during the pregnancy, which is effective by transfer of the maternal antibodies through the placenta. However, maternal antibody levels reduce rapidly and cannot provide protection to infants older than 6-8 weeks and could interfere with the active immunity induced by the primary vaccination scheme. Directly after birth, the newborns could be protected by immunization with either DTaP (vaccine for diphtheria, pertussis, and tetanus) or aP vaccine followed by the regular vaccination scheme. This approach was questioned by the reduced antibody levels against important B. pertussis toxin later in infancy. In contrast, other studies observe an increased antibody response against B. pertussis later in infancy, but reduced antibody levels for Haemophilus influenza B and hepatitis B. Based on e.g. sustainability, finance and logistical factors, the development of a new vaccine is suggested to be the best solution long-term.

To prevent worldwide outbreaks and obstruct the resurgence of pertussis, there is thus a strong need in the art for a more effective vaccine. Over the last years, increased attention goes out to the use of outer membrane vesicles (OMVs) as potential vaccine, since more knowledge is gained about their effect on immune modulation (Ellis, T. N. and M. J. Kuehn, MMBR, 2010. 74(1): p. 81-94). An advantage of OMVs as vaccine platform is their broad range of antigens, in their native conformation on the OMV surface, which can induce a protective immune response. In addition, these OMVs are equipped with self-adjuvants and can be easily taken up by immune cells. All together these properties make OMVs attractive for vaccine development (van der Pol, L., et al, Biotechnology Journal, 2015. 10(11): p. 1689-1706). Recently, vaccines composed of OMVs were developed and employed successfully, for example the OMV based Neisseria meningitidis (N. meningitidis) vaccine (Ellis, T. N., supra; FernAndez, S., et al., BMC Immunology, 2013. 14(Suppl 1): p. S8-S8). Besides injection of OMVs derived from N. meningitidis, intranasally administered OMVs induced a high and protective antibody response. Additionally, several studies have already have shown promising results for OMV based B. pertussis vaccines against pertussis (Roberts, R., et al., Vaccine, 2008.26(36): p. 4639-4646; Acevedo, R., et al., Frontiers in Immunology, 2014. 5: p. 121; Asensio, C. J. A., et al., Vaccine, 2011. 29(8): p. 1649-1656).

OMVs can be produced by Gram-negative bacteria and have a diameter of ˜20 to 250 nanometer (nm). OMVs are formed by a budding process, resulting in vesicles with the outer membrane (OM) located on their surface-exposed side, although the exact mechanism is still poorly understood. Events which are thought to play a role in the budding mechanism are a weak or missing connection between OM and peptidoglycan (PG) layer and accumulation (proteins/molecules) in the periplasmic space (van der Pol, L., supra). Additionally, the rate of OMV production differs between species, strains and even between growth phases. Production rates can be influenced by environmental factors and stress. OMVs are suggested to be ‘sample packs’ of the bacteria, which contain a large repertoire of the original biological content, but in a non-replicative form (Kaparakis-Liaskos, M. and R. L. Ferrero, Nat Rev Immunol, 2015. 15(6): p. 375-387). Biological content could comprise ribonucleic acid (RNA), Deoxyribonucleic acid (DNA), LPS, PG, enzymes and proteins including virulence factors and pathogen associated molecular patterns (PAMPs).

OMVs function in long distance delivery, biofilm formation, promote pathogenesis, bacterial survival and the regulation of interaction within bacterial communities. The presence of pertussis toxin, fimbriae 3 and pertactin has been previously confirmed in proteoliposomes derived from inactivated B. pertussis. These three virulence factors are considered to be important for the virulence of B. pertussis. OMVs are proteoliposomes which comprise of OM phospholipids and other proteins. OMVs have an advantage, since they have more in common with the actual bacterium and thereby come closer to mimicking natural infection compared to the aP vaccines used nowadays. Besides mimicking natural infection, OMVs have another advantage as they can be readily taken up by immune cells, thereby enhancing their immunogenic properties. OMVs contain LPS that could cause toxic effects in a too high dose. However, it can also function as a natural adjuvant (Raeven, R. H. M., et al., Journal of Proteome Research, 2015. 14(7): p. 2929-2942).

B. pertussis does not secrete high levels of OMVs. For the sustainability of an OMV based pertussis vaccine, the secretion rate of spontaneously formed OMVs should be optimized. Various protocols have been designed to enhance vesicle production by Gram-negative bacteria, including treatments with detergents or sonication. However, these treatments may alter the composition and properties of OMVs compared to spontaneously produced outer membrane vesicles (sOMVs). In some cases, a detergent and/or sonication free approach may thus be preferred.

There is thus a need in the art to increase OMV production in Bordetella, in particular to increase OMV vaccine production. In addition, there is still a need for a modified Bordetella having an increased blebbing phenotype. Moreover, there is a need in the art for a Bordetella having increased immunogenicity, preferably in combination with an increased blebbing phenotype.

### SUMMARY

The invention may be summarized in the following embodiments:

In an embodiment the invention pertains to a polypeptide comprising a sequence having at least 50% sequence identity with SEQ ID NO: 1 and comprises a mutation in an OmpA-like domain.

Preferably the mutation is located at a position corresponding to any one of positions 110-140 in SEQ ID NO: 1.

Preferably, the polypeptide comprising the mutation increases OMV production when expressed in Bordetella as compared to an otherwise identical polypeptide not comprising said mutation.

In an embodiment, the mutation in the polypeptide is a mutation of a single amino acid residue.

In an embodiment, the mutation in the polypeptide is a substitution of an amino acid residue, preferably a substitution at a position corresponding to position 124 of SEQ ID NO: 1, preferably a D124N substitution.

In an embodiment, the invention concerns a polynucleotide encoding the polypeptide of the invention, preferably wherein the polynucleotide has at least 50% sequence identity with SEQ ID NO: 4.

In an embodiment, the invention relates to a Bordetella bacterium comprising a genomic modification in a gene encoding a polypeptide having at least 50% sequence identity with SEQ ID NO: 1, wherein preferably the mutation is located in the open reading frame of the gene.

In an embodiment, said mutation increases OMV (Outer Membrane Vesicle) production of the Bordetella bacterium as compared to the same bacterium not comprising the mutation.

In an embodiment, the genomic modification results in the expression of a polypeptide of the invention.

In an embodiment, the mutation is in a gene comprising a sequence having at least 50% sequence identity with SEQ ID NO: 2.

In an embodiment, the Bordetella bacterium is at least one of B. pertussis, B. parapertussis and B. bronchiseptica.

In an embodiment, the Bordetella bacterium further comprises a mutation in an endogenous gene encoding LpxA.

In an embodiment, the bacterium further comprises a mutation in an endogenous gene encoding Pertactin.

In an embodiment, the bacterium further comprises a mutation in at least one of:
i) an endogenous gene encoding Ptx; and
ii) an endogenous gene encoding DNT.

In an embodiment the invention pertains to a Bordetella OMV obtainable from the Bordetella bacterium as defined herein.

In an embodiment, the Bordetella OMV of the invention comprises a polypeptide as defined herein.

In an embodiment, the invention concerns a method for producing OMVs, wherein the method comprises the steps of:
i) culturing a population of Bordetella bacteria of the invention under conditions conductive for the production of OMVs; and
ii) optionally, recovering the OMVs.

In an embodiment, the invention relates to a composition comprising at least one of:
i) a Bordetella bacterium as defined herein, wherein preferably the bacterium is an inactivated bacterium; and
ii) an OMV as defined herein.

Preferably the composition is a pharmaceutical composition.

In an embodiment, the invention concerns a composition as defined herein for use as a medicament.

In an embodiment, the composition is for use in the treatment or prevention of a Bordetella infection.

Preferably, the infection is a B. pertussis infection.

In an embodiment, the composition as defined herein or the composition for use as defined herein is an acellular or cellular vaccine.

The composition as defined herein or the composition for use as defined herein preferably further comprises at least one non-Bordetella antigen.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. Thus, for example, reference to “a cell” includes a combination of two or more cells, and the like.

As used herein, the term “about” is used to describe and account for small variations. For example, the term can refer to less than or equal to +10%, such as less than or equal to ±5%, less than or equal to +4%, less than or equal to +3%, less than or equal to +2%, less than or equal to ±1%, less than or equal to ±0.5%, less than or equal to +0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: the term “and/or” refers to a situation wherein one or more of the stated cases may occur, alone or in combination with at least one of the stated cases, up to with all of the stated cases.

“Comprising”: this term is construed as being inclusive and open ended, and not exclusive. Specifically, the term and variations thereof mean the specified features, steps or components are included. These terms are not to be interpreted to exclude the presence of other features, steps or components.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to ompA nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, aspartate-glutamate and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; arg to lys; asn to gin or his; asp to glu; cys to ser or ala; gin to asn; glu to asp; gly to pro; his to asn or gln; ile to leu or val; leu to ile or val; lys to arg; gln or glu; met to leu or ile; phe to met, leu or tyr; ser to thr; thr to ser; trp to tyr; tyr to trp or phe; and, val to ile or leu.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6×sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3rd edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or “expression construct” refer to nucleotide sequences that are capable of effecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most cells, preferably bacterial cells, under most physiological and developmental conditions. An “inducible” promoter is a promoter that is physiologically or developmentally regulated, e.g. by the application of a chemical inducer.

The term “selectable marker” is a term familiar to one of ordinary skill in the art and is used herein to describe any genetic entity which, when expressed, can be used to select for a cell or cells containing the selectable marker. The term “reporter” may be used interchangeably with marker, although it is mainly used to refer to visible markers, such as green fluorescent protein (GFP). Selectable markers may be dominant or recessive or bidirectional.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The term “peptide” as used herein is defined as a chain of amino acid residues, usually having a defined sequence. As used herein the term peptide is interchangeable with the terms “polypeptide” and “protein”. In the context of the present invention, the term “peptide” is defined as being any peptide or protein comprising at least two amino acids linked by a modified or unmodified peptide bond. The term “peptide” refers to short-chain molecules such as oligopeptides or oligomers or to long-chain molecules such as proteins. A protein/peptide can be linear, branched or cyclic. The peptide can include D amino acids, L amino acids, or a combination thereof. A peptide according to the present invention can comprise modified amino acids. Thus, the peptide of the present invention can also be modified by natural processes such as post-transcriptional modifications or by a chemical process. Some examples of these modifications are: acetylation, acylation, ADP-ribosylation, amidation and de-amidation, covalent bonding with flavine, covalent bonding with a heme, covalent bonding with a nucleotide or a nucleotide derivative, covalent bonding to a modified or unmodified carbohydrate moiety, bonding with a lipid or a lipid derivative, covalent bonding with a phosphotidylinositol, cross-linking, cyclization, disulfide bond formation, demethylation, cysteine molecule formation, pyroglutamate formation, formylation, gamma-carboxylation, hydroxylation, iodination, methylation, oxidation, phosphorylation, racemization, etc. Thus, any modification of the peptide which does not have the effect of eliminating the immunogenicity of the peptide, is covered within the scope of the present invention.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3-nontranslated sequence (3-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide. The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which it is introduced, but e.g. have been obtained from another cell or synthetically or recombinantly produced. Generally, though not necessarily, such nucleic acids encode proteins, i.e. exogenous proteins, that are not normally produced by the cell in which the DNA is transcribed or expressed. Similarly exogenous RNA may encode for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

The term “immune response” as used herein refers to the production of antibodies and/or immune cells (such as T lymphocytes) that are directed against, and/or assist in the decomposition and/or inhibition of, a particular antigenic entity, carrying and/or expressing or presenting antigens and/or antigenic epitopes at its surface. The phrases “an effective immunoprotective response”, “immunoprotection”, and like terms, for purposes of the present invention, mean an immune response that is directed against one or more antigenic epitopes of a pathogen, a pathogen-infected cell or a cancer cell so as to protect against infection by the pathogen or against cancer in a vaccinated subject. For purposes of the present invention, protection against infection by a pathogen or protection against cancer includes not only the absolute prevention of infection or cancer, but also any detectable reduction in the degree or rate of infection by a pathogen or of the cancer, or any detectable reduction in the severity of the disease or any symptom or condition resulting from infection by the pathogen or cancer in the vaccinated subject, for example as compared to an unvaccinated infected subject. An effective immunoprotective response in the case of cancer also includes clearing up the cancer cells, thereby reducing the size of cancer or even abolishing the cancer. Vaccination in order to achieve this is also called therapeutic vaccination. Alternatively, an effective immunoprotective response can be induced in subjects that have not previously been infected with the pathogen and/or are not infected with the pathogen or do not yet suffer from cancer at the time of vaccination, such vaccination can be referred to as prophylactic vaccination.

According to the present invention, the general use herein of the term “antigen” refers to any molecule that binds specifically to an antibody. The term also refers to any molecule or molecular fragment that can be bound by an MHC molecule and presented to a T-cell receptor. Antigens can be e.g. proteinaceous molecules, i.e. polyaminoacid sequences, optionally comprising non-protein groups such as carbohydrate moieties and/or lipid moieties or antigens can be e.g. molecules that are not proteinaceous such as carbohydrates. An antigen can be e.g. any portion of a protein (peptide, partial protein, full-length protein), wherein the protein is naturally occurring or synthetically derived, a cellular composition (whole cell, cell lysate or disrupted cells), an organism (whole organism, lysate or disrupted cells) or a carbohydrate or other molecule, or a portion thereof, that is able to elicit an antigen-specific immune response (humoral and/or cellular immune response) in a particular subject, which immune response preferably is measurable via an assay or method.

The term “antigen” is herein understood as a structural substance which serves as a target for the receptors of an adaptive immune response. An antigen thus serves as target for a TCR (T-cell receptor) or a BCR (B-cell receptor) or the secreted form of a BCR, i.e. an antibody. The antigen can thus be a protein, peptide, carbohydrate or other hapten that is usually part of a larger structure, such as e.g. a cell or a virion. The antigen may originate from within the body (“self”) or from the external environment (“non-self”). The immune system is usually non-reactive against “self” antigens under normal conditions due to negative selection of T cells in the thymus and is supposed to identify and attack only “non-self” invaders from the outside world or modified/harmful substances present in the body under e.g. disease conditions. Antigen structures that are the target of a cellular immune response are presented by antigen presenting cells (APC) in the form of processed antigenic peptides to the T cells of the adaptive immune system via a histocompatibility molecule. Depending on the antigen presented and the type of the histocompatibility molecule, several types of T cells can become activated. For T-Cell Receptor (TCR) recognition, the antigen is processed into small peptide fragments inside the cell and presented to a T-cell receptor by major histocompatibility complex (MHC).

The term “immunogen” is used herein to describe an entity that comprises or encodes at least one epitope of an antigen such that when administered to a subject, preferably together with an appropriate adjuvant, elicits a specific humoral and/or cellular immune response in the subject against the epitope and antigen comprising the epitope. An immunogen can be identical to the antigen or at least comprises a part of the antigen, e.g. a part comprising an epitope of the antigen. Therefore, to vaccinate a subject against a particular antigen means, in one embodiment, that an immune response is elicited against the antigen or immunogenic portion thereof, as a result of administration of an immunogen comprising at least one epitope of the antigen. Vaccination preferably results in a protective or therapeutic effect, wherein subsequent exposure to the antigen (or a source of the antigen) elicits an immune response against the antigen (or source) that reduces or prevents a disease or condition in the subject. The concept of vaccination is well-known in the art. The immune response that is elicited by administration of a prophylactic or therapeutic composition of the present invention can be any detectable change in any facet of the immune status (e.g., cellular response, humoral response, cytokine production), as compared to in the absence of the administration of the vaccine.

An “epitope” is defined herein as a single immunogenic site within a given antigen that is sufficient to elicit an immune response in a subject. Those of skill in the art will recognize that T cell epitopes are different in size and composition from B cell epitopes, and that T cell epitopes presented through the Class I MHC pathway differ from epitopes presented through the Class II MHC pathway. Epitopes can be linear sequences or conformational epitopes (conserved binding regions) depending on the type of immune response. An antigen can be as small as a single epitope, or larger, and can include multiple epitopes. As such, the size of an antigen can be as small as about 5-12 amino acids (e.g., a peptide) and as large as: a full length protein, including multimeric proteins, protein complexes, virions, particles, whole cells, whole microorganisms, or portions thereof (e.g., lysates of whole cells or extracts of microorganisms).

An adjuvant is herein understood to be an entity, that, when administered in combination with an antigen to a human or an animal subject to raise an immune response against the antigen in the subject, stimulates the immune system, thereby provoking, enhancing or facilitating the immune response against the antigen, preferably without necessarily generating a specific immune response to the adjuvant itself. A preferred adjuvant enhances the immune response against a given antigen by at least a factor of 1.5, 2, 2.5, 5, 10 or 20, as compared to the immune response generated against the antigen under the same conditions but in the absence of the adjuvant. Tests for determining the statistical average enhancement of the immune response against a given antigen as produced by an adjuvant in a group of animal or human subjects over a corresponding control group are available in the art. The adjuvant preferably is capable of enhancing the immune response against at least two different antigens.

OMV (also referred to as “blebs”) are bi-layered membrane structures, usually spherical, with a diameter in the range of 20-250 nm (sometimes 10-500 nm), that are pinched off from the outer membrane of Gram-negative bacteria. The OMV membrane contains phospholipids (PL) on the inside and lipopolysaccharides (LPS) and PL on the outside, mixed with membrane proteins in various positions, largely reflecting the structure of the bacterial outer membrane from which they pinched off. The lumen of the OMV may contain various compounds from the periplasm or cytoplasm, such as proteins, RNA/DNA, and peptidoglycan (PG), however, unlike bacterial cells, OMV lack the ability to self-replicate. In the context of the present invention three types of OMV can be distinguished depending on the method of their production. sOMV are spontaneous or natural OMV that are purified and concentrated from culture supernatant, by separating intact cells from the already formed OMVs. Detergent OMV, dOMV, are extracted from cells with detergent, such as deoxycholate, which also reduces the content of reactogenic LPS. After detergent extraction dOMV are separated from cells and cellular debris and further purified and concentrated. Finally, the term native nOMV is used herein for OMV that are generated from concentrated dead cells with non-detergent cell disruption techniques, or that are extracted from cells with other (non-disruptive) detergent-free methods (e.g. using chelating agents such EDTA), to be able to distinguish them from the wild-type spontaneous OMVs and from the detergent-extracted dOMV. A particular type of nOMV is an “eOMV”, which is used herein for OMV that are extracted from cells with the chelating agent EDTA.

Any reference to nucleotide or amino acid sequences accessible in public sequence databases herein refers to the version of the sequence entry as available on the filing date of this document.

### DETAILED DESCRIPTION OF THE INVENTION

### Polypeptide

The current invention concerns the surprising discovery that a mutation in a Bordetella ompA gene increases OMV production. Put differently, a Bordetella having a mutation in an endogenous ompA gene has a so-called high-blebbing phenotype. A Bordetella OmpA polypeptide may have a sequence as annotated in SEQ ID NO: 1.

OmpA comprises an N-terminal domain which crosses the OM with eight antiparallel 3-strands. The C-terminal domain remains in the periplasm and is suggested to interact with the PG layer (Confer, A. W. and S. Ayalew, Veterinary Microbiology, 2013. 163(3-4): p. 207-222; Mittal, R., et al., The Journal of Biological Chemistry, 2011. 286(3): p. 2183-2193). E. coli, Salmonella spp. and A. baumannii lacking OmpA display increased OMV production (Schwechheimer C. and M. J. Kuehn, Nat Rev Micro, 2015. 13(10): p. 605-619). Furthermore, deletion of the periplasmic protein RmpM in Neisseria results in increased OMV yield (Maharjan, S., et al., Microbiology, 2016. 162(2): p. 364-375; van de Waterbeemd et al, Vaccine 2010, 28 (30):4810-4516). RmpM shares limited homology with outer membrane protein A (OmpA) of i.e. Escherichia coli (E. coli) (Klugman K. P. et al, Infect Immun, 1989, 57(7):2066-71), B. pertussis and various other Gram-negative species.

Deletion of OmpA (BP0943), or any of the OmpA homologs BP2019 and BP3342 in Bordetella has been shown to be detrimental for viability (unpublished results). Hence, OmpA and its homologs appear to be essential proteins for viability in Bordetella. Surprisingly, specific mutations in Bordetella OmpA provide for viable bacteria having a high-blebbing phenotype.

In a first aspect, the invention pertains to a mutant OmpA polypeptide, i.e. an OmpA polypeptide having a mutation. Preferably, the invention concerns a polypeptide comprising a sequence having at least about 50% sequence identity with SEQ ID NO: 1 and wherein said polypeptide comprises a mutation.

Preferably, the polypeptide comprising the mutation increases OMV production when expressed in Bordetella, as compared to an otherwise identical polypeptide not comprising said mutation. Preferably, the Bordetella expressing of the OmpA polypeptide comprising a mutation as defined herein has an increased OMV production as compared to an otherwise identical Bordetella bacterium expressing an endogenous OmpA polypeptide. Preferably, the endogenous OmpA polypeptide is identical to the polypeptide comprising the mutation, except that the endogenous polypeptide does not comprise the mutation.

The mutant polypeptide comprising the mutation may comprise a sequence having at least about 55%, 60%, 65%, 70%, 75%, 80%, 85%, 90%, 95%, 96%, 97%, 98%, 99% or at least about 100% sequence identity with SEQ ID NO: 1. Preferably, the polypeptide may comprise a sequence having SEQ ID NO: 1, with the exception of a mutation as defined herein.

The mutant polypeptide comprising the mutation may comprise a sequence having at least about 55%, 60%, 65%, 70%, 75%, 80%, 85%, 90%, 95%, 96%, 97%, 98%, 99% or at least about 100% sequence identity with a sequence having NCBI reference sequence NP879744.1. Preferably, the polypeptide may comprise a sequence having NCBI reference sequence NP879744.1, with the exception of a mutation as defined herein.

The mutant polypeptide may be a mutant of an orthologue or a mutant of an paralogue of an OmpA polypeptide having SEQ ID NO: 1, preferably the mutant polypeptide may be a mutant of an orthologue of an OmpA polypeptide. Preferably, the orthologue is an B. parapertussis OmpA or an B. bronchiseptica OmpA. Preferably, the B. parapertussis OmpA has at least about 55%, 60%, 65%, 70%, 75%, 80%, 85%, 90%, 95%, 96%, 97%, 98%, 99% or at least about 100% sequence identity with SEQ ID NO: 8, with the exception of a mutation as defined herein. Preferably, the mutation is at a position corresponding to position D124 in SEQ ID NO: 1. Preferably, the B. bronchiseptica OmpA has at least about 55%, 60%, 65%, 70%, 75%, 80%, 85%, 90%, 95%, 96%, 97%, 98%, 99% or at least about 100% sequence identity with SEQ ID NO: 9, with the exception of a mutation as defined herein. Preferably, the mutation is at a position corresponding to position D124 in SEQ ID NO: 1.

The mutant polypeptide may comprise a sequence having at least about 55%, 60%, 65%, 70%, 75%, 80%, 85%, 90%, 95%, 96%, 97%, 98%, 99% or at least about 100% sequence identity with SEQ ID NO: 3.

Preferably the mutation is located in an OmpA-like domain of the polypeptide. Preferably, the OmpA-like domain has at least about 75%, 80%, 85%, 90%, 95%, 96%, 97%, 98%, 99% or about 100% sequence identity with positions 75-191 of SEQ ID NO: 1. The polypeptide therefore preferably has a mutation at a position corresponding to any one of positions 75-191 of SEQ ID NO: 1.

The mutation may be located at a position corresponding to any one of positions 110-140 in SEQ ID NO: 1. Preferably, the mutation may be located at a position corresponding to any one of positions 80-180, 85-170, 90-160, 95-150, 100-140, 110-140, 110-130, 115-130, 120-128 or any one of positions 122-126 in SEQ ID NO: 1. The mutation may be located at a position corresponding to any one of positions 120, 121, 122, 123, 124, 125, 126, 127, 128, 129 and 130 in SEQ ID NO: 1, preferably any one of positions 122, 123, 124, 125, 126 in SEQ ID NO: 1. Preferably, the mutation is located at a position corresponding to position 124 in SEQ ID NO: 1.

Preferably, the OmpA polypeptide comprising the mutation is an endogenous protein, except for the mutation as defined herein. Hence preferably, the OmpA polypeptide is derivable from a Bordetella bacterium, and wherein the OmpA polypeptide further comprises a mutation as defined herein.

In an embodiment, the mutation is a mutation of at least 1, 2, 3, 4, 5, 6, 7, 8, 9 or 10 amino acid residues in a sequence having at least 50% sequence identity with SEQ ID NO: 1. Preferably, the mutation is a mutation of 1, 2, 3, 4 or 5 amino acid residues in a sequence having at least about 50% sequence identity with SEQ ID NO: 1. Preferably, the mutation is a mutation of a single amino acid residue in a sequence having at least 50% sequence identity with SEQ ID NO: 1.

The mutation can be any one of a deletion, addition and substitution of one or more amino acid residues. Preferably, the mutation is at least one of a deletion, addition and substitution of a single amino acid residue. Preferably, the mutation is a substitution of a single amino acid residue. The mutation is preferably a substitution of an amino acid residue that corresponds to position 124 in SEQ ID NO: 1. Preferably, the mutation is a substitution of an aspartic acid amino acid residue at a position corresponding to position 124 in SEQ ID NO: 1.

The amino acid residue substitution can be a conservative or a non-conservative amino acid residue substitution. A conservative amino acid residue substitution is defined herein as substituting an amino acid residue for a different amino acid residue having similar biochemical properties, such as having at least one of a similar charge, hydrophobicity and size.

Preferably an acidic residue may be substituted for a different acidic residue. Preferably the acidic residue is selected from the group consisting of Aspartate, Glutamate, Asparagine and Glutamine.

Preferably a basic residue may be substituted for a different basic residue. Preferably the basic residue is selected from the group consisting of Histidine, Lysine and Arginine.

Preferably an aliphatic residue may be substituted for a different aliphatic residue. Preferably the aliphatic residue is selected from the group consisting of Glycine, Alanine, Valine, Leucine and Isoleucine.

Preferably a hydroxyl or sulphur/selenium containing residue may be substituted for a different hydroxyl or sulphur/selenium containing residue. Preferably the hydroxyl or sulphur/selenium containing residue is selected from the group consisting of Serine, Cysteine, Selenocysteine, Threonine and Methionine.

Preferably an aromatic residue may be substituted for a different aromatic residue. Preferably the aromatic residue is selected from the group consisting of Phenylalanine, Tyrosine and Tryptophan.

In the mutant polypeptide of the invention preferably an Aspartate, preferably an Aspartate at a position that corresponds to position 124 in SEQ ID NO: 1, may be substituted for at least one of Glutamate, Asparagine and Glutamine. Preferably an Aspartate, preferably an Aspartate at a position that corresponds to position 124 in SEQ ID NO: 1, may be substituted for Asparagine (i.e. a D to N substitution).

Preferably, the mutant polypeptide of the invention has at least about 50% 55%, 60%, 65%, 70%, 75%, 80%, 85%, 90%, 95%, 96%, 97%, 98%, 99% or at least about 100% sequence identity with SEQ ID NO: 3.

Preferably, the polypeptide is isolated from its natural environment. The polypeptide can be a recombinant, synthetic or artificial polypeptide.

### Polynucleotide

In a second aspect, the invention pertains to a polynucleotide encoding a polypeptide as defined herein. Preferably, the polynucleotide encodes a polypeptide having at least about 50%, 55%, 60%, 65%, 70%, 75%, 80%, 85%, 90%, 95%, 97%, 98%, 99% or 100% sequence identity with SEQ ID NO: 1, with the exception of a mutation as defined herein. Part or all of the codons may be optimized for expression in bacteria, preferably for expression in Bordetella, preferably for expression in Bordetella as defined herein. The codons may be identical to the codons of an endogenous Bordetella ompA coding sequence, with the exception of the mutation as defined herein. The polynucleotide may be preceded by an endogenous promoter, preferably an endogenous promoter driving the expression of OmpA in Bordetella. Preferably, the polynucleotide is isolated from its natural environment. The polypeptide can be a recombinant, synthetic or artificial polynucleotide. The polynucleotide may comprise one or more nucleotides that are not present in a naturally occurring Bordetella OmpA-encoding polynucleotide.

Preferably, the polynucleotide has one or more nucleotides that are not present in a naturally occurring Bordetella OmpA-encoding polynucleotide.

Preferably, the polynucleotide has at least about 55%, 60%, 65%, 70%, 75%, 80%, 85%, 90%, 95%, 96%, 97%, 98%, 99% or at least about 100% sequence identity with SEQ ID NO: 4.

Preferably, the polynucleotide has at least about 55%, 60%, 65%, 70%, 75%, 80%, 85%, 90%, 95%, 96%, 97%, 98%, 99% or at least about 100% sequence identity with SEQ ID NO: 2, with the exception of a mutation as defined herein above. Preferably, the polynucleotide has at least about 55%, 60%, 65%, 70%, 75%, 80%, 85%, 90%, 95%, 96%, 97%, 98%, 99% or at least about 100% sequence identity with SEQ ID NO: 2, with the exception of a mutation at position 370, 371 and/or 372, i.e. the codon corresponding to position 124 of SEQ ID NO: 1.

In a third aspect, the invention concerns a gene encoding the polypeptide as defined in the first aspect. Preferably, the gene comprises a polynucleotide as defined in the second aspect. The gene may comprise elements regulating the expression of a OmpA polypeptide. Preferably, the gene comprises a promoter controlling the expression of a mutated (OmpA) polypeptide as defined herein. The promoter can be a constitutively active promoter or an inducible promoter. The promoter can be an endogenous promoter driving the expression of OmpA in Bordetella.

In a fourth aspect, the invention relates to vector comprising at least one of a polynucleotide of the second aspect and a gene of the third aspect. The vector is preferably suitable for transformation into bacteria, preferably suitable for transformation into Bordetella. Preferably, the vector is a DNA plasmid, preferably a naked DNA plasmid.

### Bordetella

In a fifth aspect, the invention pertains to a genetically modified Bordetella. Preferably, the genetically modified Bordetella has an increased OMV production as compared to an otherwise identical Bordetella not comprising the mutation, preferably when grown under identical conditions. Preferably, the genetically modified Bordetella comprises and/or expresses a modified polypeptide as defined herein. Preferably, the genetically modified Bordetella does not comprise an endogenous OmpA polypeptide.

In an embodiment, the Bordetella of the invention comprises a modification, preferably a genomic modification, wherein the modification results in the expression of a mutant OmpA polypeptide as defined herein. Preferably, the modified Bordetella does not express an endogenous OmpA polypeptide.

The modification can be the insertion of a vector as defined herein. The vector may remain episomal or may be inserted into the bacterial genome. The modification may be the insertion of at least one of a polynucleotide and a gene as defined herein into the Bordetella genome. The modified Bordetella may further comprise a modification that reduces or abolishes the expression of an endogenous OmpA polypeptide. Preferably, the genetically modified Bordetella may comprise a genomic modification that reduces or abolishes the expression of an endogenous OmpA polypeptide, preferably a genomic modification in the OmpA gene. Preferably, the mutation in the OmpA gene is a genomic modification in a regulatory element controlling the expression of an endogenous OmpA polypeptide or a mutation in the OmpA polypeptide coding sequence.

The modification may be the insertion of a suicide vector, preferably the suicide vector pSS1129 (Stibitz, S., Use of conditionally counterselectable suicide vectors for allelic exchange. Methods Enzymol, 1994. 235: p. 458-65). The insertion of the suicide vector results in the expression of a mutant OmpA polypeptide, and a lack of expression of an endogenous OmpA polypeptide.

The modification can be a modification of the Bordetella genome, wherein the modification modifies the coding sequence of the endogenous OmpA polypeptide, resulting in the expression of a mutant OmpA polypeptide as defined herein. Modification of a genomically encoded OmpA polypeptide can be done using any suitable means known to the person skilled in the art. Preferably, the mutation is located in the coding sequence and/or preferably the mutation results in the expression of a mutant OmpA polypeptide as defined herein.

Preferably the genetically modified Bordetella bacterium of the invention comprises a genomic modification in a sequence encoding a polypeptide having at least about 35%, 40%, 45%, 50%, 55%, 60%, 65%, 70%, 75%, 80%, 85%, 90%, 95%, 96%, 97%, 98%, 99% or at least about 100% sequence identity with SEQ ID NO: 1, wherein the mutation results in the expression of a mutant polypeptide that increases OMV production. Preferably, the mutation results in the expression of a mutant polypeptide as defined herein.

Preferably the genetically modified Bordetella bacterium of the invention comprises a genomic modification in a coding sequence having at least about 35%, 40%, 45%, 50%, 55%, 60%, 65%, 70%, 75%, 80%, 85%, 90%, 95%, 96%, 97%, 98%, 99% or at least about 100% sequence identity with SEQ ID NO: 2, wherein the mutation results in the expression of a mutant polypeptide that increases OMV production.

Preferably, the mutation results in the expression of a mutant polypeptide as defined herein.

Preferably the genetically modified Bordetella bacterium of the invention comprises a genomic modification in a gene encoding a polypeptide having at least about 35%, 40%, 45%, 50%, 55%, 60%, 65%, 70%, 75%, 80%, 85%, 90%, 95%, 96%, 97%, 98%, 99% or at least about 100% sequence identity with SEQ ID NO: 8, wherein the mutation increases OMV production.

Preferably the genetically modified Bordetella bacterium of the invention comprises a genomic modification in a gene encoding a polypeptide having at least about 35%, 40%, 45%, 50%, 55%, 60%, 65%, 70%, 75%, 80%, 85%, 90%, 95%, 96%, 97%, 98%, 99% or at least about 100% sequence identity with SEQ ID NO: 9, wherein the mutation increases OMV production.

Preferably OMV production is increased as compared to an otherwise identical or substantially identical Bordetella not comprising the mutation as defined herein. OMV production is understood herein at least one of sOMV (spontaneous or natural OMV) production, dOMV (detergent OMV) production and nOMV (native OMV) production. Preferably OMV production as defined herein refers to at least one of sOMV and dOMV production.

Preferably, the OMV production is increased at least about 1.5 fold, or at least 2-, 3-, 4-, 5-, 6-, 7-, 8-, 9-, 10-, 20-, 30-, 40-, 50-, 60-, 70-, 80-, 90- or at least about 100-fold. The increase in OMV production may be dependent on the Bordetella strain comprising the mutation as defined herein.

Preferably, the spontaneous or supernatant (sOMV) production or yield is increased at least about 1.5 fold, or at least about 2-, 3-, 4-, 5-, 6-, 7-, 8-, 9-, 10-, 20-, 30-, 40-, 50-, 60-, 70-, 80-, 90- or at least about 100-fold. In addition or alternatively, the detergent OMV (dOMV) production or yield is increased at least about 1.5 fold, or at least about 2-, 3-, 4-, 5-, 6-, 7-, 8-, 9-, 10-, 20-, 30-, 40-, 50-, 60-, 70-, 80-, 90- or at least about 100-fold. In addition or alternatively, the native OMV (nOMV) production or yield is increased at least about 1.5 fold, or at least about 2-, 3-, 4-, 5-, 6-, 7-, 8-, 9-, 10-, 20-, 30-, 40-, 50-, 60-, 70-, 80-, 90- or at least about 100-fold. In addition or alternatively, the EDTA-extracted (eOMV) production or yield is increased at least about 1.5 fold, or at least about 2-, 3-, 4-, 5-, 6-, 7-, 8-, 9-, 10-, 20-, 30-, 40-, 50-, 60-, 70-, 80-, 90- or at least about 100-fold.

The OMV production of a Bordetella may be determined using any conventional method known in the art. As a non-limiting example, the OMV yield may be determined using an FM4-64 assay.

The modified bacterium is preferably a Bordetella is preferably selected from the group consisting of Bordetella ansorpii, Bordetella avium, Bordetella bronchialis, Bordetella bronchiseptica, Bordetella flabilis, Bordetella hinzii, Bordetella holmesii, Bordetella muralis, Bordetella parapertussis, Bordetella pertussis, Bordetella petrii, Bordetella pseudohinzii, Bordetella sputigena, Bordetella trematum, Bordetella tumbae and Bordetella tumulicola. Preferably, the Bordetella for use in the invention is at least one of Bordetella pertussis, Bordetella parapertussis and Bordetella bronchiseptica. Preferably, the modified Bordetella is Bordetella pertussis In a preferred embodiment, the modified Bordetella is Bordetella pertussis. Preferably, the genetically modified bacterium is a B. pertussis Tohama I strain or a derivative thereof. Preferably, the derivative Tohama I strain is a streptomycin-resistant derivative of the Tohama I strain and most preferably the genetically modified bacterium is derived from the strain B213 or a derivative thereof. Alternatively, the genetically modified bacterium is a B. pertussis B1917 or B1920 strain or a derivative thereof.

In addition or alternatively, the modified Bordetella as defined herein may comprise at least one of the following mutations:
A mutation that results in expression of a mutant OmpA polypeptide as defined herein;
A mutation that results in the retention of Prn93 (93 kDa Pertactin) in the outer membrane;
A mutation that results in heterologous acyl transferase activity;
A mutation that results in detoxification of pertussis toxin (Ptx); and
A mutation that reduces or abolishes expression of dermonecrotic toxin (DNT).

In an embodiment, the modified Bordetella as defined herein may comprise the following mutations:
A mutation that results in expression of a mutant OmpA polypeptide as defined herein; and
A mutation that results in the retention of Prn93 (93 kDa Pertactin) in the outer membrane.

In an embodiment, the modified Bordetella as defined herein may comprise the following mutations:
A mutation that results in expression of a mutant OmpA polypeptide as defined herein; and
A mutation that results in heterologous acyl transferase activity.

In an embodiment, the modified Bordetella as defined herein may comprise the following mutations:
A mutation that results in expression of a mutant OmpA polypeptide as defined herein; and
A mutation that results in detoxification of pertussis toxin (Ptx).

In an embodiment, the modified Bordetella as defined herein may comprise the following mutations:
A mutation that results in expression of a mutant OmpA polypeptide as defined herein; and
A mutation that reduces or abolishes expression of dermonecrotic toxin (DNT).

In an embodiment, the modified Bordetella as defined herein comprises the following mutations:
A mutation that results in expression of a mutant OmpA polypeptide as defined herein;
A mutation that results in the retention of Prn93 (93 kDa Pertactin) in the outer membrane;
A mutation that results in heterologous acyl transferase activity;
A mutation that results in detoxification of pertussis toxin (Ptx); and
A mutation that reduces or abolishes expression of dermonecrotic toxin (DNT).
   Pertactin (93 kDa)

In an embodiment, the modified Bordetella as defined herein further comprises a mutation that results in the retention of Prn93 (93 kDa Pertactin) in the outer membrane, preferably resulting in the retention of Prn93 in the OMV.

Pertactin (Prn) is a known protective antigen. Pm is an autotransporter that is cleaved off by B. pertussis during growth. The modified Bordetella as defined herein preferably comprises a mutation that prevents autocatalytic cleavage, resulting in retention of full-length Pm (93 kDa) in OMVs instead of shedding of Pm (69 kDa) in the environment.

The Bordetella Pertactin polypeptide preferably has at least about 35%, 40%, 45%, 50%, 55%, 60%, 65%, 70%, 75%, 80%, 85%, 90%, 95%, 96%, 97%, 98%, 99% or at least about 100% sequence identity with SEQ ID NO: 5 and comprises a mutation that prevents autocatalytic cleavage. Preferably, the mutation is at a position that corresponds to a position D738 of SEQ ID NO: 5. Preferably, the mutation is a substitution of a conservative amino acid residue. Preferably, the mutation is a substitution of Asp (D) to Asn (N), preferably a D738N mutation. Preferably, the mutated Bordetella Pertactin polypeptide has at least about 35%, 40%, 45%, 50%, 55%, 60%, 65%, 70%, 75%, 80%, 85%, 90%, 95%, 96%, 97%, 98%, 99% or at least about 100% sequence identity with SEQ ID NO: 11.

Preferably, the retention of Prn93 in the OMV increases the protective immunity of the OMV. The inventors discovered that retaining Prn93 in the outer membrane of the OMV surprisingly resulted in a significant increase in immunogenicity of the OMV, e.g. when compared to the immunogenicity of an OMV in combination with the same amount of (purified) Pm. Preferably, the protective immunity of an OMV comprising Prn93 in its outer membrane is increased at least about 1.5-, 2-, 2.5-, 3-, 3.5- or at least about 4-fold as compared to the protective immunity of a same or similar OMV not comprising Prn93 combined with a same or similar amount of (purified) Pertactin. The protective immunity may be determined using any conventional means known in the art. Preferably, the protective immunity is determined by intranasal challenge after vaccination of mice and determining the anti-Prn antibody concentrations.

### Acyl Transferase

In an embodiment, the modified Bordetella as defined herein further comprises heterologous acyl transferase activity. Hence, the modified Bordetella preferably further comprises a modification that introduces heterologous acyl transferase activity. Preferably, the modification is a modification as described in WO2018/167061, which is incorporated herein by reference.

The modification that introduces heterologous acyl transferase activity may confer to the cell at least one of a heterologous LpxA and heterologous LpxD acyl transferase activity.

Preferably the modification introduces the expression of at least one of a heterologous IpxA and a heterologous IpxD gene. Preferably the modification introduces the expression of at least a heterologous IpxA gene. Preferably, the modification is a genomic modification.

Preferably, the heterologous IpxA gene has a nucleotide sequence that encodes a LpxA acyl transferase that has at least about 60%, 65%, 70%, 75%, 80%, 85%, 90%, 95%, 96%, 97%, 98%, 99% or at least about 100% amino acid sequence identity with SEQ ID NO: 6. The IpxA gene may be obtained or is obtainable from the genus Pseudomonas, preferably from species Pseudomonas aeruginosa.

Preferably, the heterologous IpxD gene has a nucleotide sequence that encodes a LpxD acyl transferase that has at least about 60% 65%, 70%, 75%, 80%, 85%, 90%, 95%, 96%, 97%, 98%, 99% or at least about 100% amino acid sequence identity with SEQ ID NO: 7. The IpxD gene may be obtained or is obtainable from the genus Pseudomonas, preferably the species Pseudomonas aeruginosa.

Preferably, the modified Bordetella further comprises a genomic modification that reduces or eliminates the activity of LpxA and/or LpxD acyl transferase encoded by respectively an endogenous IpxA gene and/or an endogenous IpxD gene.

Introducing heterologous acyltransferase activity into Bordetella has been shown in the art to reduce LPS endotoxicity. The inventors now discovered that the introduction of a heterologous LpxA acyl transferase in a Bordetella bacterium also increases OMV production, i.e. results in a high-blebbing phenotype. In particular, heterologous acyl transferase activity as defined herein surprisingly increases the production of at least one of sOMV and nOMV.

### Pertussis Toxin (Ptx)

Although Pertussis toxin is secreted and thus not part of OMVs, small amounts may remain in or on the OMVs. Therefore in an embodiment, the modified Bordetella as defined herein further comprises a mutation that reduces or eliminates Pertussis toxin (Ptx) toxicity. Preferably, the mutation results in a reduced or eliminated toxicity of a Pertussis toxin having at least about 80%, 85%, 90%, 95%, 96%, 97%, 98%, 99% or 100% sequence identity with SEQ ID NO: 5.

Any suitable method known in the art for partly of fully detoxifying Ptx is equally suitable for use in the invention. As a non-limiting example, one or more mutations may be introduced into the Ptx gene, which mutations result in a partly or fully detoxified Ptx. These one or more mutations can be point mutations, such as but not limited to, point mutations that may result in the inactivation of an enzymatic site in the subunit 1 of Ptx. Such mutation may prevent or reduce, among others, leucocytosis in mice. Preferred mutations are located at R43 and/or E163, preferably position 43 and/or position 163 of the amino acid sequence SEQ ID NO: 12, preferably a R43K and/or E163G mutation.

### Dermonecrotic Toxin (DNT)

A Bordetella OMV may comprise small amounts of the cytoplasmic protein dermonecrotic toxin (DNT) and residual DNT may contribute to the reactogenecity of an OMV. DNT was given its name because it may cause necrotic skin lesions when injected subcutaneously in mice. In an embodiment, the modified Bordetella as defined herein further comprises a mutation that results in the reduced or complete absence of DNT expression. Preferably, the DNT has a sequence having at least about 80%, 85%, 90%, 95%, 96%, 97%, 99% or 100% sequence identity with SEQ ID NO: 10.

As a non-limiting example, dnt gene expression may be reduced by knocking down or knocking out the gene encoding DNT, such as, but not limited to genomic modification of an element controlling the expression of DNT and/or genomic modification of the sequence encoding DNT.

### Outer Membrane Vesicle (OMV)

In a further aspect, the invention pertains to a Bordetella OMV obtainable or obtained from a modified Bordetella as defined herein.

In an aspect, the invention pertains to an OMV comprising a modified polypeptide as defined herein, preferably the OMV is a Bordetella OMV.

OMVs (also known as “blebs”), e.g. for use in vaccines, have traditionally been prepared by detergent extraction (a dOMV purification process), wherein detergents such as deoxycholate are used to remove LPS and increase vesicle release. An OMV preparation, prepared by sonication of cells and treatment with DOC, combined with alum adjuvant provided protection against pertussis challenge in a mouse model [Roberts, R., Vaccine 2008, 26, 4639-4646], which was comparable to the effect of a whole-cell vaccine.

Another version of OMVs containing a PagL-deacylated modified LPS showed both protection and a lower reactogenicity, the latter determined in vivo by both weight gain and cytokine induction [Asensio, C. J., Vaccine 2011, 29, 1649-1656]. Another interesting finding with B. parapertussis OMVs was their cross-protection against both pertussis and parapertussis [Bottero, D. Vaccine 2013, 31, 5262-5268].

Above a certain threshold the wildtype LPS of Bordetella can be toxic and detergents may be used to remove wild type LPS, e.g. during the OMV extraction process. Alternatively or in addition, LPS may be modified to reduce the endotoxicity. Therefore in an embodiment, the Bordetella and/or OMV of the invention comprises a modified LPS having reduced toxicity. As a non-limiting example, the modified LPS having reduced endotoxicity may be obtained by introducing heterologous acyltransferase activity in the Bordetella, such as described in WO2018/167061. The modified LPS obtained from such modified Bordetella preferably has a lipid A moiety that is modified as compared to the lipid A moiety of a wild-type Bordetella LPS in that the length of at least one acyl chain is shorter. Preferably, the length of the acyl chain at the 3 position of the modified lipid A moiety does not have a greater length than the acyl chain of the wild-type Bordetella lipid A moiety at the same 3 position, preferably the length of the acyl chain at the 3 position of the modified lipid A moiety is not greater than C₁₀, wherein more preferably the length of the acyl chain at the 3 position of the modified lipid A moiety has the same length as the acyl chain of the wild-type Bordetella lipid A moiety at the same 3 position, and preferably the length of the acyl chain at the 3 position is C₁₀. Preferably, the shorter acyl chain is selected from the group consisting of:
i) the acyl chain at the 3′ position of the lipid A moiety;
ii) the primary acyl chain at the 2′ position of the lipid A moiety;
iii) the secondary acyl chain at the 2′ position of the lipid A moiety; and
iv) the acyl chain at the 2 position of the Lipid A moiety.

In addition or alternatively, the Bordetella of the invention has, or has an increased, 3-O-deacylase activity, such as e.g. described in WO/2006/065139, which is incorporated herein by reference. Preferably, such modified Bordetella comprises 3-O-deacylated LPS.

Bordetella LPS having a reduced endotoxicity may remain present in the OMV at a higher concentration than the toxic wild-type LPS. The OMV as defined herein therefore may be obtainable by detergent extraction of by spontaneous release from the Bordetella.

A preferred OMV comprising the Bordetella of the invention can be a supernatant or spontaneous OMV, i.e. an sOMV as herein defined above, or a native OMV, i.e. an nOMV as herein defined above. Alternatively the OMV is a detergent-extracted OMV, i.e. an dOMV as defined herein above.

In a further aspect, the invention concerns a method for producing an OMV, preferably an OMV as defined herein. Preferably, the method comprises a step of i) culturing a population of Bordetella bacteria as defined herein under conditions conductive for the production of OMVs; and optionally, recovering the OMVs.

Methods for preparing dOMV, sOMV and nOMV are described in van de Waterbeemd et al (2010) and van de Waterbeemd et al (2013) (van de Waterbeemd B et al, Vaccine. 2010; 28(30):4810-6 and van de Waterbeemd B., PLoS One. 2013 31; 8(5):e65157) and WO2013/006055, all of which are incorporated herein by reference.

The method for producing an OMV may be any conventional detergent extraction method. Alternatively, the extraction method can be a detergent-free extraction method, e.g. as described in WO/2013/006055. It is understood herein that a process for producing an OMV that is not a detergent-extracted OMV, does not exclude the use of low concentration of detergent and/or the use of mild detergents.

The OMV production of a modified Bordetella as defined herein is increased as compared to the OMV production of an otherwise identical Bordetella not comprising a modification as defined herein. Preferably, the OMV production is increased at least about 1.5 fold, or at least 2-, 3-, 4-, 5-, 6-, 7-, 8-, 9-, 10-, 20-, 30-, 40-, 50-, 60-, 70-, 80-, 90- or at least about 100-fold.

Preferably, the spontaneous or supernatant (sOMV) production or yield is increased at least about 1.5 fold, or at least about 2-, 3-, 4-, 5-, 6-, 7-, 8-, 9-, 10-, 20-, 30-, 40-, 50-, 60-, 70-, 80-, 90- or at least about 100-fold. In addition or alternatively, the detergent OMV (dOMV) production or yield is increased at least about 1.5 fold, or at least about 2-, 3-, 4-, 5-, 6-, 7-, 8-, 9-, 10-, 20-, 30-, 40-, 50-, 60-, 70-, 80-, 90- or at least about 100-fold. In addition or alternatively, the native OMV (nOMV) production or yield is increased at least about 1.5 fold, or at least about 2-, 3-, 4-, 5-, 6-, 7-, 8-, 9-, 10-, 20-, 30-, 40-, 50-, 60-, 70-, 80-, 90- or at least about 100-fold. In addition or alternatively, the EDTA-extracted (eOMV) production or yield is increased at least about 1.5 fold, or at least about 2-, 3-, 4-, 5-, 6-, 7-, 8-, 9-, 10-, 20-, 30-, 40-, 50-, 60-, 70-, 80-, 90- or at least about 100-fold.

### Compositions

In an aspect, the invention concerns a composition comprising at least one of a modified Bordetella as defined herein and an OMV as defined herein.

Preferably, the composition is a pharmaceutical composition. The pharmaceutical composition may comprise a pharmaceutically acceptable excipient, carrier, medium or delivery vehicle conventionally known in the art (see e.g. “Handbook of Pharmaceutical Excipients”, Rowe et al eds. 7th edition, 2012, www.pharmpress.com). Pharmaceutically acceptable stabilizing agents, osmotic agents, buffering agents, dispersing agents, and the like may also be incorporated into the pharmaceutical compositions. The preferred form of the composition depends on the intended mode of administration and therapeutic application. The pharmaceutical carrier can be any compatible, non-toxic substance suitable to deliver to the patient.

Pharmaceutically acceptable carriers for parenteral delivery are exemplified by sterile buffered 0.9% NaCl or 5% glucose optionally supplemented with a 20% albumin. Alternatively, the active ingredients of the invention can be suspended in Phosphate buffered saline (PBS). Preparations for parenteral administration must be sterile. The parenteral route for administration of the active ingredients of the invention is in accord with known methods, e.g. injection or infusion by intravenous, intraperitoneal, intramuscular, and intra-arterial or intralesional routes. Alternatively, the composition maybe administrated by inhalation. The composition may be administrated continuously by infusion or by bolus injection. Preferably, the composition is administrated by bolus injection. A typical pharmaceutical composition for intramuscular injection would be made up to contain, for example, 1-10 ml of phosphate buffered saline comprising the effective dosages of the active ingredients of the invention. Methods for preparing parenterally administrable compositions are well known in the art and described in more detail in various sources, including, for example, “Remington: The Science and Practice of Pharmacy” (Ed. Allen, L. V. 22nd edition, 2012, www.pharmpress.com). The “active ingredients of the invention” are herein understood to be at least one of a modified Bordetella and an OMV as defined herein.

The modified Bordetella as defined herein may be an attenuated or inactivated Bordetella. The Bordetella may be inactivated using any conventional method known in the art for inactivating the bacterium, such as, but not limited to further genomically modifying the Bordetella, chemical treatment or thermally inactivating the Bordetella. A preferred chemical inactivation is formaldehyde-treatment.

The composition may further comprise at least one additional antigen, preferably at least 1, 2, 3, 4, 5, 6, 7, 8, 9 or 10 additional antigens. Preferably, the composition may further comprise at least one additional non-Bordetella antigen, preferably at least 1, 2, 3, 4, 5, 6, 7, 8, 9 or 10 additional non-Bordetella antigens.

The composition of the invention may further comprise 1, 2, 3 or more antigens of the bacterium of the genus Bordetella. The composition may further comprise inactivated Bordetella toxin either alone or in combination with other Bordetella components such as filamentous haemagglutinin, fimbrial antigens and pertactin.

It is understood herein that these components may be separately added to the composition comprising at least one of the modified Bordetella and OMV, or one or more of these components are part of the modified Bordetella and OMV in the composition.

The composition may further comprise one or more adjuvants. The adjuvant may be present in at least one of the modified Bordetella and OMV. Alternatively or in addition, an (additional) adjuvant may be added to the composition comprising at least one of the modified Bordetella and OMV. The adjuvant may be an organic or inorganic adjuvant. A preferred inorganic adjuvant is an aluminium salt, such as, but not limited to aluminium phosphate and aluminium hydroxide. A preferred organic adjuvant may be a modified LPS, preferably modified Neisserial or Bordetella LPS, modified LOS, squalene, QS21, or monophosphoryl lipid A (MPL).

The composition as defined herein may be a Bordetella vaccine.

### Medical Uses

In an aspect, the invention pertains to a composition comprising at least one of a modified Bordetella as defined herein and an OMV as defined herein for use as a medicament. Put differently, the invention thus pertains to the use as medicament of at least one of a modified Bordetella of the invention, an OMV of the invention, and a pharmaceutical composition of the invention. The invention further concerns a method of treatment using at least one of the modified Bordetella, an OMV and a pharmaceutical composition as defined herein.

In a further aspect, the invention relates to a composition comprising at least one of the modified Bordetella and an OMV as defined herein for use in a treatment comprising inducing an immune response in a subject. Alternatively, the invention relates to a composition comprising at least one of the modified Bordetella and an OMV as defined herein for use in a treatment comprising stimulating an immune response in a subject. In particular, the invention thus relates to a method for vaccination. Preferably, the immune response is induced or stimulated against a Bordetella infection.

In an aspect, the invention pertains to a composition as defined herein for use in the treatment or prevention of a Bordetella infection. To this end, three Bordetella species are known human pathogens (B. pertussis, B. parapertussis and B. bronchiseptica). The Bordetella infection is therefore preferably at least one of a B. pertussis, B. parapertussis and B. bronchiseptica infection, preferably a Bordetella pertussis infection.

B. pertussis and occasionally B. parapertussis cause pertussis or whooping cough in humans, and some B. parapertussis strains can colonise sheep. B. bronchiseptica rarely infects healthy humans, though disease in immunocompromised patients has been reported. B. bronchiseptica causes several diseases in other mammals, including kennel cough and atrophic rhinitis in dogs and pigs, respectively. Other members of the genus cause similar diseases in other mammals, and in birds (B. hinzii, B. avium).

Most preferably, the immune response is induced or stimulated against a Bordetella pertussis infection. In a further preferred embodiment, the invention pertains to a composition as defined herein for use in a treatment or prevention of whooping cough. To this end, the subject is unvaccinated or may have been previously vaccinated against Bordetella. It is further noted that the terms “whooping cough”, “pertussis” and “100-day cough” may be used interchangeable herein.

In a preferred embodiment, the pharmaceutical composition of the invention is a vaccine. The vaccine can be an acellular vaccine preferably comprising an OMV as defined herein. Alternatively, the vaccine is a whole cell vaccine comprising at least a modified Bordetella as defined herein.

The invention pertains to a (pharmaceutical) composition for use in the treatment or prevention of a Bordetella infection, wherein the composition is a whole cell vaccine comprising a modified Bordetella as defined herein. The modified Bordetella of the invention may be a live or live attenuated bacterium or non-viable bacterium. Preferably, the bacterium is inactivated or killed using means known in the art per se. For example, the modified Bordetella may have been inactivated by freezing, heat treatment, mechanical disruption, chemical treatment or other methods known in the art of pharmacy and vaccination (see e.g.

J. L. Pace, H. A. Rossi, V. M. Esposito, S. M. Frey, K. D. Tucker, R. I. Walker. Inactivated whole-cell bacterial vaccines: current status and novel strategies. Vaccine 16: 1563-1574 (1998)). Preferably the bacterium is a Bordetella pertussis, Bordetella parapertussis or Bordetella bronchiseptica and most preferably a Bordetella pertussis.

In an alternatively preferred embodiment, the (pharmaceutical) composition according to the invention is an acellular vaccine comprising an OMV as defined herein.

In another embodiment, the invention pertains to a composition as defined herein for use as a medicament, or for use in a treatment comprising inducing or stimulating an immune response in a subject, wherein the composition further comprises at least one non-Bordetella antigen. The antigen is any antigen as defined herein. In particular, a Bordetella vaccine may be combined with other vaccines known in the art. In a preferred embodiment the Bordetella vaccine is combined with at least one of a diphtheria and tetanus vaccine. In an embodiment, the Bordetella vaccine is combined with a diphtheria as well as a tetanus vaccine.

Pharmaceutically acceptable composition and vaccines according to the invention may be used in methods of treatment of subjects suffering from or at risk of acquiring a Bordetella infection, comprising administering at least one of the pharmaceutical composition, a whole cell and an a-cellular vaccine according to the invention. The use of specific adjuvants, the relative and absolute amounts of substances in the compositions and the doses regimen for the administration are known or may be determined by the skilled person and may be adapted for the circumstances such as the particular pathogenic infection or the status of the particular subject to be treated. The doses regimen may comprise a single dose but may also comprise multiple doses, for instance booster doses and may preferably be administered orally, intranasally or parenterally. Various doses regimens for vaccination purposes are known in the art and may be suitably adapted by the skilled person.

### Further Aspects

In an aspect, the invention pertains to a process for producing a modified Bordetella or OMV of the invention. The process preferably comprises the steps of a) cultivating a modified Bordetella as defined herein; and optionally b) at least one of purifying and inactivating the modified Bordetella. In addition to step b), or instead of step b), the OMV may be extracted and/or purified. Methods for purifying and inactivating Bordetella are well-known in the art. Similarly, the purifying/extraction of OMV can be performed using any suitable method known in the art.

In an aspect, the invention relates to producing a vaccine formulation comprising at least one of an, preferably inactivated, modified Bordetella and OMV as defined herein. The process preferably comprises the steps of a) cultivating a modified Bordetella as defined herein; b) at least one of purifying and inactivating the genetically modified bacterium and c) formulating at least one of the modified Bordetella and OMV, optionally with further vaccine components, into a vaccine formulation. In addition to step b), or instead of step b), the OMV may be extracted and/or purified prior to step c).

It is further understood that the use of the composition in treatments of medical conditions as specified herein also includes the use of the compositions for the manufacture of a medicament for the corresponding medical treatments, as well as, methods for treating a subject suffering from such medical conditions by administering an effective amount of the compositions to the subject.

In an aspect, the invention pertains to a Bordetella, preferably a Bordetella pertussis, comprising a mutation that results in the retention of Prn93 (93 kDa Pertactin) in the outer membrane. The Bordetella may further comprise at least one of the following mutations:
A mutation that results in expression of a mutant OmpA polypeptide as defined herein;
A mutation that results in heterologous acyl transferase activity;
A mutation that results in detoxification of pertussis toxin (Ptx); and
A mutation that reduces or abolishes expression of dermonecrotic toxin (DNT).

In an embodiment, the invention pertains to an OMV obtainable from said Bordetella, wherein preferably said OMV has an increased or improved immunogenicity, e.g. when compared to the immunogenicity of an OMV in combination with the same amount of (purified) Pm.

In a further embodiment, the invention pertains to the production, use and a composition comprising at least one of the OMV and the Bordetella, preferably as defined herein above for a modified Bordetella comprising an OmpA mutation.

In an aspect, the invention pertains to a Bordetella, preferably a Bordetella pertussis, comprising a mutation that results in heterologous acyl transferase activity. The modified Bordetella preferably expresses a protein having at least about 50%, 55%, 60%, 65%, 70%, 75%, 80%, 85%, 90%, 95%, 97%, 98%, 99% or 100% sequence identity with SEQ ID NO: 6 (LpxA) or SEQ ID NO: 7 (LpxD). The Bordetella may further comprise at least one of the following mutations:
A mutation that results in expression of a mutant OmpA polypeptide as defined herein;
A mutation that results in the retention of Prn93 (93 kDa Pertactin) in the outer membrane;
A mutation that results in detoxification of pertussis toxin (Ptx); and
A mutation that reduces or abolishes expression of dermonecrotic toxin (DNT).

In an embodiment the invention pertains to a method for producing OMVs, wherein the method comprises a step of culturing a population of Bordetella bacteria, wherein the Bordetella bacteria have a heterologous acyl transferase activity as defined herein, under conditions conductive for the production of OMVs, and optionally, recovering the OMVs. Preferably, the method for producing OMVs is the same or is similar as the method for producing OMVs from a modified Bordetella comprising an OmpA mutation, as specified herein above.

In a further embodiment, the invention pertains to the production, use and a composition comprising at least one of the OMV and the Bordetella, preferably as defined herein above for a modified Bordetella comprising an OmpA mutation.

In an aspect, the invention pertains to a Bordetella, preferably a Bordetella pertussis, comprising a mutation that results in detoxification of pertussis toxin (Ptx). The Bordetella may further comprise at least one of the following mutations:
A mutation that results in expression of a mutant OmpA polypeptide as defined herein;
A mutation that results in the retention of Prn93 (93 kDa Pertactin) in the outer membrane;
A mutation that results in heterologous acyl transferase activity; and
A mutation that reduces or abolishes expression of dermonecrotic toxin (DNT).

In a further embodiment, the invention pertains to the production, use and a composition comprising at least one of the OMV and Bordetella, preferably as defined herein above for a modified Bordetella comprising an OmpA mutation.

In an aspect, the invention pertains to a Bordetella, preferably a Bordetella pertussis, comprising a mutation that reduces or abolishes expression of dermonecrotic toxin (DNT). The Bordetella may further comprise at least one of the following mutations:
A mutation that results in expression of a mutant OmpA polypeptide as defined herein;
A mutation that results in the retention of Prn93 (93 kDa Pertactin) in the outer membrane;
A mutation that results in heterologous acyl transferase activity; and
A mutation that reduces or abolishes expression of dermonecrotic toxin (DNT).

In further embodiment, the invention pertains to the production, use and a composition comprising at least one of the OMV and Bordetella, preferably as defined herein above for a modified Bordetella comprising an OmpA mutation.

All patent and literature references cited in the present specification are hereby incorporated by reference in their entirety.

The following examples are offered for illustrative purposes only, and are not intended to limit the scope of the present invention in any way.

TABLE 1
Sequence identifiers

| SEQ ID NO. | Description |
|---|---|
| 1 | Bordetella pertussis OmpA a.a.(wt) |
| 2 | Bordetella pertussis OmpA nt. (wt) |
| 3 | Bordetella pertussis OmpA D124N mutation a.a. |
| 4 | Bordetella pertussis OmpA D124N mutation nt. |
| 5 | Bordetella pertussis Pertactin a.a. (wt) |
| 6 | LpxA Pseudomonas aeruginosa a.a. |
| 7 | LpxD Pseudomonas aeruginosa a.a. |
| 8 | Bordetella parapertussis OmpA a.a. |
| 9 | Bordetella bronchiseptica OmpA a.a. |
| 10 | Bordetella pertussis DNT a.a. |
| 11 | Bordetella pertussis Pertactin D738N mutation a.a. |
| 12 | Bordetella pertussis toxin (wt) |

### FIGURE LEGEND

FIG. 1. Schematic overview of the sOMV isolation from B. pertussis cultures. sOMV were isolated after approximately 30h from 200 ml cultures grown under standard lab conditions, unless otherwise stated. 22 pm sterile filtered supernatant, centrifuged+22 μm sterile filtered supernatant and purified sOMV preparations were obtained from this sOMV isolation process. Samples were stored at 4° C. prior to sOMV characterizing experiments

FIG. 2. sOMV concentration of isolated sOMV preparations obtained from bacterial cultures of B. pertussis B1917 (Wt), B1917/OmpA-D124N, B1917/BP2019-D50N and B1917/OmpA-D124N/BP2019-D50N. Bacterial cultures grew for approximately 30h at 35° C. with 200 rpm. sOMV were isolated from 200 ml cultures and concentrated into 1 ml. Concentration sOMV determined by a FM4-64 assay were corrected for the concentration factor to obtain the original sOMV concentration, ****P>0.0001. The grey bounding line indicates two separate grown bacterial cultures.

FIG. 3. FIG. 3. Influence of stress on sOMV secretion by B. pertussis B1917/Wt, B1917/OmpA-D124N, B1917/BP2019-D50N and B1917/OmpA-D124N/BP2019-D50N. sOMV concentrations were determined by FM4-64 assay directly in sterile filtered supernatant samples that were simultaneously collected with the samples to measure the OD and pH. B1917 (Wt), B1917/OmpA-D124N, B1917/BP2019-D50N and B1917/OmpA-D124N/BP2019-D50N were grown in 200 ml medium for approximately 30h at 35° C. with 200 rpm, with or without stress treatment ****P<0.0001. A) sOMV secretion under stress conditions based on sterile filtered supernatant after approximately 30h of growth and B) sOMV secretion under non-stress conditions based on sterile filtered supernatant after approximately 30h of growth.

FIG. 4. sOMV concentration of isolated sOMV preparations obtained from bacterial cultures of B. pertussis B213 (Wt), B213/PagL-KI, B213/BP2329-KO and B213/OmpA-D124N. Bacterial cultures grew for approximately 30h at 35° C. with 200 rpm. sOMV were isolated from 200 ml cultures and concentrated into 1 ml. Concentration sOMV determined by a FM4-64 assay were corrected for the concentration factor to obtain the original sOMV concentration, **P<0.01, ***P<0.001.

FIG. 5. Protein concentration determined in isolated sOMV preparations obtained from bacterial cultures of B. pertussis B213 mutants compared to the corresponding wild-type and OmpA-D124N mutant. Bacterial cultures grew for approximately 30h at 35° C. with 200 rpm. sOMV were isolated from 200 ml cultures and concentrated into 1 ml. protein concentration was determined by a BCA assay and were corrected for the original volume of the bacterial culture, *P>0.05, **P>0.01, ****P>0.0001. protein concentrations measured by BCA in sOMV preparations of B213 (Wt), B213/PagL-KI, B213/BP2329-KO and B1917/OmpA-D124N.

FIG. 6. LPS concentration normalized to 25 μg of purified sOMV secreted by B. pertussis B213/PagL-KI and B213/BP2329-KO in comparison to the corresponding wild-type and OmpA-D124N mutant. Bacterial cultures grew for approximately 30h at 35° C. with 200 rpm. sOMV were isolated from 200 ml cultures and concentrated into 1 ml. LPS concentration was determined by the phenol sulfuric acid method, normalized to 25 ug protein and corrected for the concentration factor to obtain the original LPS concentration per 25 μg protein. *P>0.05, ***P<0.001. LPS concentration normalized to 25 μg sOMV protein from sOMV preparations of B213 (Wt), B213/PagL-KI, B213/BP2329-KO and B213/OmpA-D124N.

FIG. 7. LpxA mutant increases OMV production. A culture volume of 50 ml was centrifuged at 3000 rpm for 30 minutes to separate the sOMV from the biomass. The supernatant, which contains the sOMV, was sterile filtered by a Nalgene vacuum system and processed by an ultracentrifugation (UC) step at 125.000×g for 90 minutes at 4° C. Finally, the sOMV pellet was re-suspended in 2.5 ml end buffer (0.01 M Tris +3% sucrose pH 7.4). The pelleted biomass of the culture after the centrifugation step was further processed to eOMV. The pellet of the harvest was re-suspended in 9 ml 0.1M Tris buffer pH 8.6 containing 0.1M EDTA and incubated for 30 minutes at room temperature while stirring to extract the eOMV. After the incubation time the suspension was transferred to UC tubes and by high speed centrifugation at 23.500×g for 15 minutes at 4° C. the cells were separated from the eOMV. The supernatant, containing the eOMV was sterile filtered by a Nalgene vacuum system and again spin down by UC at 125.000×g for 90 minutes at 4° C. to pellet the eOMV. The eOMV pellet was finally re-suspended in 2.5 ml end buffer. Both, the sOMV and eOMV fractions were analysed for the total protein concentration by Peterson, the DNA concentration by PicoGreen, the protein pattern by SDS-PAGE, protein composition by MS and the OMV size by DLS. The yield is shown as number of OMV particles per volume.

FIG. 8. Increase of protective immunity after retaining of Pm to the outer membrane of the OMV. Mice were immunized with OMVs at days 0 and 14, and challenged with B. pertussis at day 28. A) Lung colonization. Lung colonization was analysed at day 35. Regression analysis of lung colonization after infection of mice, immunized twice (s.c.) with 16, 4 or 1 μg OMV-WT (grey line) and 16, 4 or 1 μg OMV-Prn93 (black line). Lung colonization after immunization with Prn-OMV is significantly decreased with a factor 4.0 (2.4-6.3) compared to WT-OMV (A). B) Anti Pm antibody response. Adding 3 μg purified Pm to 1 human dose (HD) of omv-WT increases the anti-Prn antibody response 10 times but retaining the same amount of Pm to the outer membrane of 1 HD of omv-Prn increases the anti-Prn antibody response a 100 times compared to omv-WT only.

### EXAMPLES

### 1. Strain History

Bordetella pertussis B1917 (B.p. B1917) is a clinical isolate from a three-year old Dutch patient with whooping cough (Mooi, F. R., et al., Bordetella pertussis strains with increased toxin production associated with pertussis resurgence. Emerg Infect Dis, 2009. 15(8): p. 1206-13). In order to make B.p. B1917 amenable for genetic engineering with counterselectable suicide vector pSS1129 (Stibitz, S, supra), a mutant with resistance to streptomycin (Strep®) was isolated. This mutant was subsequently used to isolate a mutant with resistance to nalidixic acid (Nal®) and this clone served as the starting material for the strain construction described below.

### 2. Strain Construction

### 2.1 Brief Overview of Strain Construction

Using genetic engineering, several mutations were introduced in the B.p. B1917 genome;
1. OmpA-D124N: amino acid change D124N in Outer membrane protein A (OmpA).
2. Prn-D738N: amino acid change D738N in Pertactin (Prn).
3. LpxA^{Pa}: replacement of LpxA with its homologue from Pseudomonas aeruginosa.
4. PtxA-R43K-E163G: amino acid changes R43K and E163G in Pertussis Toxin subunit 1 (PtxA).
5. Deletion of the coding sequence of Dermonecrotic Toxin (Dnt).

A detailed overview of the mutations can be found in Table 2.

TABLE 2
Overview of mutations introduced in B.p. B1917 (Strep^{R}, Nal^{R}).

| | | Observed | Mutation in B.p. | Codon | Amino acid |
|---|---|---|---|---|---|
| Mutation | Nature | effect | B1917 genome¹ | change | change² |
| OmpA-D124N | Amino acid | Increases OMV | g3,092,828a | GAC → AAC | D124N |
| | change | formation | | | |
| Prn-D738N | Amino acid | Prevents | g2,972,475a | GAC → AAC | D738N |
| | change | autocleavage of | | | |
| | | pertactin | | | |
| LpxA^{Pa} | Gene | LPS detoxification; | Replacement of LpxA | N/A | N/A |
| | replacement | increases OMV | (2,574,474-2,573,704) | | |
| | | formation | with LpxA from | | |
| | | | P. aeruginosa | | |
| | | | (GenBank NC_002516: | | |
| | | | 4,082,961-4,082,182) | | |
| PtxA-R43K- | Two amino | Detoxification of | c4,004,381a | CGC → AAG | R43K |
| E163G | acid changes | pertussis toxin | g4,004,382a | | |
| | | subunit 1 (PtxA) | c4,004,383g | | |
| | | | a4,004,742g | GAA → GGC | E163G |
| | | | a4,004,743c | | |
| ΔDnt | Deletion | Removal of | Deletion of 3,419,774-3,424,168 | N/A | N/A |
| | | dermonecrotic toxin | | | |

¹Coordinates are based on the B.p. B1917 genome sequence (GenBank accession number: CP009751). Nucleotide mutations are shown as ‘original nucleotide - coordinate in CP009751- new nucleotide’, e.g. ‘g3,092,828a’ indicates that the guanidine residue at position 3,092,828 was changed to an adenine residue.
²Amino acid changes are shown as ‘original amino acid - residue number - new amino acid’, using the IUPAC single letter code.

2.2 Genetic Engineering of B.p. B1917 Using Counterselectable Suicide Vector pSS1129

All mutations were introduced in the genome of B. pertussis B1917 (Strep®, Nal®) using counterselectable suicide vector pSS1129 (Stibitz, S, supra). Constructs (and primers needed for their construction) were first designed in silico using SnapGene (GSL Biotech, Chicago, USA). Constructs for OmpA-D124N, Prn-D738N, PtxA-R43K-E163G, and ADnt were created with overlap extension PCR (Horton, R. M., et al., Engineering hybrid genes without the use of restriction enzymes: gene splicing by overlap extension. Gene, 1989. 77(1): p. 61-8), for which primers were ordered at Eurofins MWG (Ebersberg, Germany). Construct LpxA^{Pa} was designed in silico and then synthesized and cloned in pUC57 by GenScript (Nanjing, China). Details of the specific PCR- and cloning-procedures are described for each construct separately in the paragraphs below.

All constructs were eventually cloned in suicide vector pSS1129 and then transformed in E.c. SM10. The pSS1129 plasmid with construct can be transferred from E.c. SM10 to B.p. B1917 (Strep®, Nal®) by conjugation, which results in the uptake of linear plasmid by B.p. B1917. Because of the homology between the construct on the plasmid and the B.p. B1917 genome, this may result in homologous recombination and uptake of the full plasmid in the B.p. B1917 genome. Cells that have integrated the plasmid in their genome can be selected because they are resistant to ampicillin/gentamycin and sensitive to streptomycin (only genomic recombinants survive, because pSS1129 cannot replicate as a plasmid in B.p. B1917). After genomic uptake of the plasmid, cells contain both the construct designed to introduce a genomic change in a certain gene as well as the wildtype version of this gene.

Successful first crossover clones are plated on streptomycin, so that spontaneous streptomycin resistant clones can be obtained. These clones are usually the result of a crossover between the introduced construct and the wildtype version of the targeted gene.

If the second recombination takes place on the other side of the introduced mutation as the first recombination, the recombinant carries the mutated version of the gene. Successful incorporation of the desired mutations(s) in second crossover clones is verified by PCR. The detailed cloning procedure for OmpA is outlined below. The other mutations depicted in Table 2 are produced using similar standard molecular biology techniques known in the art. The mutations were introduced in strain B213 as well as strain B1917.

### 2.2.1 OmpA—Introduction of Amino Acid Substitution D124N

Several amino acids in OmpA were substituted and the effect of these mutations on OMV formation was studied. An R139A as well as an R139L substitution in B. pertussis OmpA appeared to be lethal (data not shown). In addition, complete knock-out of OmpA, or knock-out of either one of the OmpA homologs BP2019 and BP3342 resulted in lethality.

It was found that the substitution D124N surprisingly resulted in viable cells and increased OMV formation. Notably, the same substitution at the corresponding location (D50N) in the homolog BP2019 resulted in viable cells, but did not have any effect on OMV production, and the substitution at the corresponding location (D100N) in the homolog BP3342 appeared to be lethal.

The mutagenesis construct for the introduction of the point mutation that causes amino acid change D124N in OmpA was created by overlap extension PCR (Horton, R. M., et al., supra). Genomic DNA of B.p. B1917 was used as template for PCR Ia with primer pair B1917-OmpA-Fw/OmpA-D124N-Rv and PCR Ib with primer pair OmpA-D124N-Fw/B1917-OmpA-Rv (see Table 3 for primer sequences). Primers OmpA-D124N-Fw and OmpA-D124N-Rv contain a point mutation compared to the B.p. B1917 genome, roughly halfway each primer. As a consequence, PCR products Ia and Ib both contain the same mismatch to the B.p. B1917 genome. A mix of PCR products Ia and Ib was used as template for PCR II with primer pair B1917-OmpA-Fw/B1917-OmpA-Rv. The resulting amplicon is a copy of the region 3,092,271-3,093,392 (GenBank CP009751), except for mutation g3,092,828a that causes amino acid change D124N in OmpA.

The PCR II amplicon was ligated in linear pGEM-T Easy Vector (Promega) using TA-cloning, resulting in pGEM-T Easy+PCR II. After amplification in E.c. JM109, the plasmid was digested with EcoRI and a 1007 bp band was purified and then ligated into EcoRI digested pSS1129. The resulting pSS1129+OmpA-D124N was subsequently transformed in E.c. SM10 cells and successful transformants were stored as a glycerol stock.

Plasmid pSS1129+OmpA-D124N was transferred from E.c. SM10 to B.p. B1917 Nal® Strep® by conjugation. A two-step antibiotic selection procedure was then used for incorporation of the mutation in the B.p. genome. Two successful second crossover clones identified by sequencing were stored as a glycerol stock.

TABLE 3
Primers used for overlap extension PCR and sequencing of OmpA-D124N.

| Primer name | Sequence (5′−>3′) | Used for | SEQ ID NO: |
|---|---|---|---|
| B1917-OmpA- | ggtcaatgcaacggtctagg | Overlap extension PCR (PCRs | 13 |
| Fw | | Ia and II) | |
| OmpA-D124N- | gccgatcgagttcgtgtggccaac | Overlap extension PCR (PCR | 14 |
| Rv | | Ia) | |
| OmpA-D124N- | ttggccacacgaactcgatcgg | Overlap extension PCR (PCR | 15 |
| Fw | | Ib) | |
| B1917-OmpA- | atgctctccgacaggatg | Overlap extension PCR (PCRs | 16 |
| Rv | | Ib and II) | |
| OmpA-seq-Fw | cgtatgtaaggatgaacc | Sequencing | 17 |
| OmpA-seq-Rv2 | tgttcgagcatttccatg | Sequencing | 18 |

Bold: codon change compared to B.p. B1917 genome

Bold and underlined: nucleotide change compared to B.p. B1917 genome

2.3 Analysis of sOMV-Secreting Properties by B. pertussis and Mutants

B. pertussis and mutants were screened towards their growth performance and sOMV secreting properties. Optical density (590 nm) and pH were measured at certain time points (T) to characterize the growth performances of, and sOMV secretion by various B213 and B1917 mutants compared to the corresponding wildtype. A variety of assays were used to quantify and characterize the sOMV secreted by B. pertussis and mutants into the supernatant. sOMV concentration was quantified, based on the lipid content by a N-(3-Triethylammoniumpropyl)-4-(6-(4-(Diethylamino) Phenyl) Hexatrienyl) Pyridinium Dibromide (FM4-64) assay. A second method was used for sOMV quantification, a Bicinchoninic acid (BCA) assay, while the size of the sOMVs was measured by Dynamic Light Scattering (DLS). The LPS concentration of the sOMV was determined by the phenol sulfuric method. Finally the variety of different proteins within the secreted sOMVs were established by sodium dodecyl sulfate polyacrylamide gel electrophoresis (SDS-PAGE).

2.3.1. Inoculation and Culturing of B. pertussis and Mutants

A visible amount of bacteria, collected by a sterile cotton swap, grown in BG agar plates, supplemented with 300 μg/ml Strep was used for the inoculation of a pre-culture. Pre-cultures could also be inoculated by previous prepared seedlots. This pre-culture was subsequently used to inoculate the main culture (MC). B. pertussis and mutants were grown in THIJS medium, supplemented with 1% THIJS supplement. Pre-cultures were always grown in 50 ml medium in a 125 ml PBF (plain bottom flask), while the main culture was grown in 50 ml (125 ml PBF), 100 ml (250 ml baffled flask) or in 200 ml (500 ml PBF). Bacterial cultures were grown at 350° C., 200 rpm unless stated otherwise.

### Standard Lab Conditions (Non-Stress Treatment)

Bacterial cultures were grown under standard conditions in THIJS medium, supplemented with 1% THIJS supplement. Cultures were grown at 35° C., 200 rpm without any form of stress. MC was inoculated with a pre-culture resulting in an OD of 0.05, unless stated otherwise.

### Stress Treatment

As stress is known to be an important factor which may influence the growth and sOMV secreting properties in a variety of bacterial species, a stress treatment is given to bacterial cultures of B. pertussis and mutants to examine the influence of stress on the growth and sOMV secreting properties. The stress treatment was consisting of temperature fluctuations and lack of oxygen as result by collecting samples for OD (590 nm) and pH measurements, as well as the sample collection for subsequent sOMV quantification. Samples for sOMV quantification during growth were sterile filtered over 0.22 μM filters. As control for the stress treatment, an exact same bacterial culture of the corresponding strain will grow parallel under standard lab conditions. The OD and pH value of these control cultures were only measured at the start and end of the growth period of approximately 30h, even as the collection of a sterile filtrated supernatant sample for subsequent sOMV quantification

### 2.3.2. Collection of Different Processed Samples

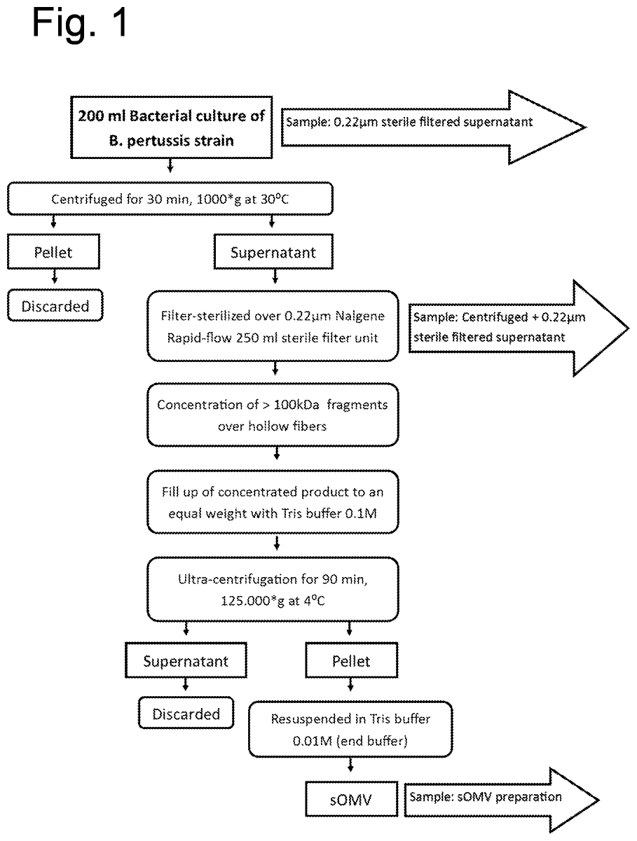

Samples were collected at the beginning and the end of culturing and in case of the performance of a growth curve also in between of the growth period. The sOMV concentration of these samples were compared to each other and to the concentration of actual isolated sOMVs in order to determine the representativeness of the different processed samples for the actual secreted sOMVs by B. pertussis and mutants. Moment of collecting the various samples in the process is displayed in (FIG. 1). Samples were stored at 40° C. prior to use for sOMV quantification and characterization experiments.

### 0.22 μM Sterile Filtered Supernatant

At the start and at the end of a growth period, samples were collected from the bacterial culture and filtered over 0.22 μM filters (Millex-GV Syringe Filter Unit) in order to remove cells. In case of OD (590 nm) measurements to characterize the growth properties in combination with screening the sOMV secreting properties during growth, 0.22 μM sterile filtered samples were collected every 2 a 3 hours. Samples were stored at 40° C. prior to sOMV quantification experiments to ensure the stability of present sOMV.

Centrifugation in Combination with 0.22 μM Sterile Filtration

At the end of a growth period, the bacterial culture was centrifuged for 30 min at 1000*g and subsequently sterile filtered over a 0.22 μM Nalgene® Rapid-Flow™ 250 ml sterile filter unit. Samples were stored at 40° C. prior to sOMV quantification experiments to ensure the stability of present sOMV.

Isolated and Purified sOMVs

At the end of a growth period, secreted sOMV were isolated (section 2.3.4.). Samples were stored at 40° C. prior to sOMV quantification experiments to ensure the stability of present sOMV.

2.3.3. Measurement of Growth Properties of B. pertussis and Mutants

B. pertussis and mutants were screened for their growth characteristics by OD (590 nm) measurements. In addition, also the pH value was determined. These measurements were performed at certain time points to characterize their growth properties over time. The pH was monitored as it can influence the growth rate and can introduce stress. Bacteria were cultured in either 50 or 200 ml medium. Cultures were either inoculated by a pre-culture or seedlots.

2.3.4. sOMV Isolation

MCs (200 ml) of B. pertussis strains were grown under standard lab conditions, unless stated otherwise. Bacterial cultures were harvested after approximately 30h for sOMV isolation. A schematic overview of the isolation protocol is displayed in FIG. 1. The mass of the culture was documented as well as the products obtained after the sOMV isolation steps in order to calculate sOMV concentration of the original bacterial culture. The culture was centrifuged for 30 min at 1000*g, 20° C. The obtained pellet was discarded and the supernatant was subsequently sterile filtered over a 0.22 μm Nalgene® Rapid-Flow™ 250 ml sterile filter unit. >100 kilodalton (kDa) fragments were concentrated over hollow fibers, mPES MicroKros® Filter Modules, into approximately 50 ml. The concentrated products were filled up to an equal weight by Tris buffer, consisting of 0.1M Tris; pH8.6 before ultra-centrifugation (UC) for 90 min at 125.000*g, 40° C. The generated pellet, assumed to contain the sOMVs was resuspended in end buffer, consisting of 0.01M Tris; pH 7.4. sOMV preparation were stored at 4° C. to ensure the stability of the sOMV preparations which were subsequent used in sOMV quantification experiments.

### 2.3.5. Lipid Concentration Determined by FM4-64 Assay

FM4-64 is a dye which give a fluorescence signal upon incorporation into a lipid environment, hereby the concentration of membrane content can be determined and thereby the sOMV concentration as described in the art. The membrane content in supernatant is assumed to be the result of sOMV production by B. pertussis and mutants. A standard curve was prepared with a OMV stock solution (B1917WT) The stock solution contains a known concentration of OMV stimulated by detergent (based on protein content), derived from B. pertussis B1917 wild-type strain. The stock solution was diluted in THIJS medium, resulting in a standard curve with a range between 0.31 μg-10 μg per ml. THIJS medium and milliQ or PBS were included as negative controls. 50 μl of the standard, controls and samples were added in triplicates to a black 96 well micro titer plate (Greiner 655209 black flat bottom). FM4-64 dye was prepared by the dilution of FM4-64 (250 μM) into MilliQ, with a final concentration of 5 μM. 50 μl of the prepared FM4-64 dye was added to each well and the fluorescence signal was measured immediately at 645 nm, with excitation at 485 nm (protocol: FM4-64 Synaptored 485 645). Concentration of sOMVs μg/ml was calculated by using the equation of the standard curve.

### 2.3.6. Protein Concentration Determined by BCA Assay

Pierce BCA protein assay (Thermo Scientific) is a colorimetric assay which is commonly used to determine the total protein concentration, also in supernatant. The protein content in a sample was assumed to be the result of sOMV production by B. pertussis and mutants. This assay uses the copper (Cu⁺²) to Cu⁺¹ reduction as result of the protein in an alkaline medium, the biuret reaction. The Cu⁺¹ is detected by the reagent containing BCA. Two BCA molecules are binding to one Cu⁺¹ molecule resulting in a color switch from light blueish to purple. A standard curve is prepared with Bovine serum albumin (BSA) with a range between 25 μg-500 μg per ml. The absorbance was measured at 562 nm. The actual concentration of protein was calculated by using the equation of the standard curve.

### 2.3.7. LPS Concentration Determined by the Phenol Sulfuric Acid Method

LPS is known as natural adjuvant, enhancing the cellular immune response by stimulating B-cell development, but also to trigger T cells to produce interferon gamma (IFN-γ) and tumor necrosis factor TNF. Lipid A is the major element of LPS which is responsible for the adjuvant effect. While LPS can have a stimulating effect on the immune system, LPS can also be toxic in too high concentrations. The commonly used KDO assay to determine the LPS concentration was not an option for B. pertussis LPS as B. pertussis LPS only contain a single KDO molecule. The phenol sulfuric acid method was used as alternative for LPS determination. This colorimetric method is used to measure the carbohydrate concentration within the sOMV, which is assumed in the art to be related to the LPS concentration]. To determine the LPS concentration within sOMVs, a standard was prepared from LPS with a known concentration of 0.39 mg/ml, isolated from N. meningitidis Lpxl1 mutant and a serial dilution was prepared with a concentration range between 0.003 mg-0.39 mg per ml. 50 μl of isolated sOMV preparation, as well as 50 μl of the standard were added to micronic tubes in duplicate. 150 μl of sulfuric acid was added, followed by 30 μl of 5% phenol. Subsequently the tubes were heated at 900° C. for 5 min and afterwards cooled down for 5 min at RT. 200 μl was transferred to a flat bottom 96-wells plate and the OD was measured at 490 nm. Concentration of LPS in the sOMV was calculated by using the equation of the standard curve.

### 2.3.8. Dynamic Light Scattering

The size (d/nm) of the isolated sOMVs was determined by DLS. 100 μl of the sOMV preparation was added to the curvet and placed in the Malvern, Zen 3600 from the Zetasizer Nano Series equipped with a with 633-nm red laser. The size was measured at 25° C. 2.3.9. Variety of proteins determined by SDS-PAGE SDS-page was performed to examine the variety of proteins within isolated sOMV, 10 μl of Lane Marker Reducing sample buffer (5×) (Thermo scientific) containing 3M Tris⋅HCl, 5% SDS, 50% glycerol, 100 mM dithiothreitol (DTT) and proprietary pink tracking dye, was added to 40 μl sample. The mixed sample was heated at 100° C. for 10 minutes and 10-15 μl was loaded to the wells of a NuPAGE Novex 4-12% Bis-Tris gel and run with 1% (2-(N-morpholiono)ethanesulfonic acid (MES) buffer at a constant 200 Volt for 35 minutes. Novex® Sharp Pre-stained Protein Standard was included as marker. The gel was stained with Coomassie blue (Imperial protein stain, Thermo Scientific) for 2 h and de-stained in milliQ until the Coomassie blue background disappeared. The gel was scanned and the contrast was adapted to create clear and visible bands.

### 2.3.10. Statistics

Analysis of variance (ANOVA) was used to determine significant differences between different samples in comparison to the control. If a group of samples were compared to the overall control Tukeys test was used while Dunnett's test was used if a significant difference was examined among all individual samples. If a comparison was made only between two samples, a unpaired T test was applied. Only if P<0.05, a significant difference was considered between the mutants compared to the control or different collected samples.

### 3. Results.

Analysis of sOMV-producing properties of newly created B. pertussis B1917 mutants and in advanced created B. pertussis B213 LPS mutants
3.1 sOMV Concentration

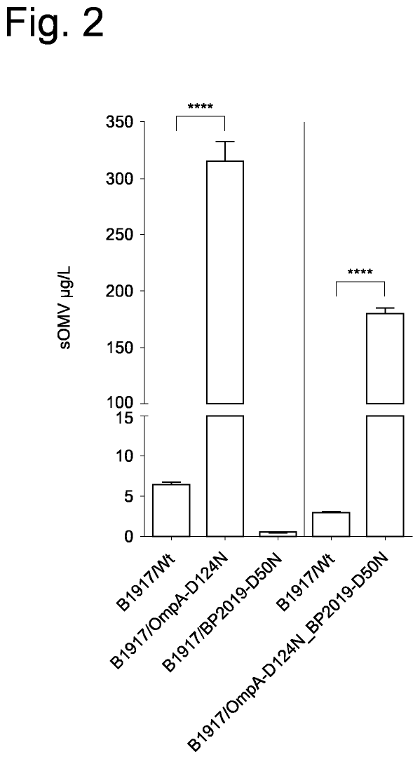

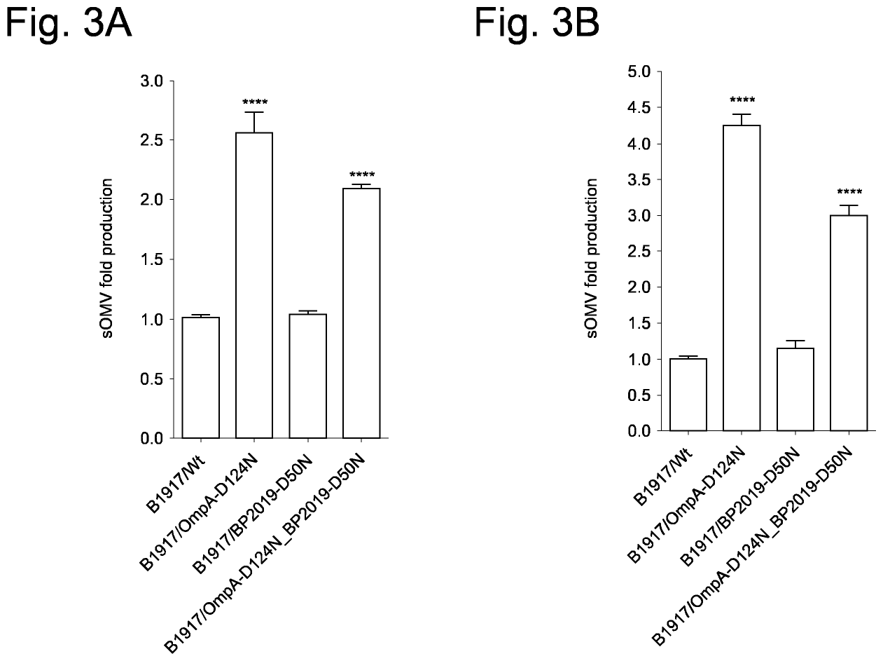

The sOMV concentration of isolated sOMV preparations was determined by FM4-64 assay and corrected for the concentration factor to calculate the sOMV concentration of the original bacterial culture (FIG. 2). B1917/OmpA-D124N did show an increased sOMV secretion (315.21 μg/L±12.23) compared to the B1917 (Wt) (0.02 μg/L±0.10) with P<0.0001. (FIG. 3 and data not shown). A significant increase was also observed for B1917/OmpA-D124N/BP2019-D50N (178.48 μg/L±6.98) compared to the B1917 (Wt) (2.88 μg/L±0.11) with P<0.0001. No increased sOMV concentration was observed in the isolated sOMV preparation of B1917/BP2019-D50N (FIG. 3).

3.2 sOMV Secreting Properties of B213/PagL-KI and B213/BP2329-KO During Growth in Comparison to the B213 (Wt) and B213/OmpA-D124N

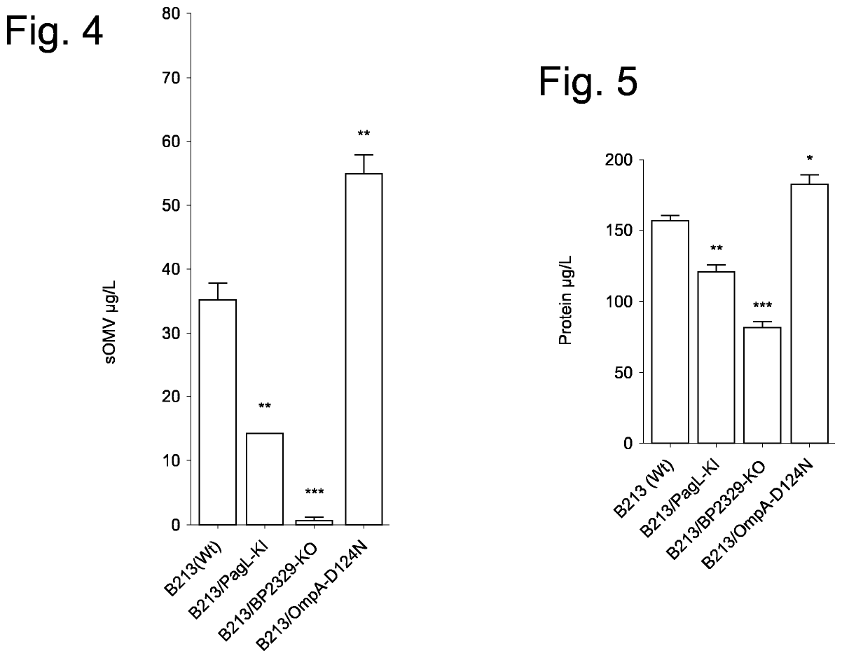

sOMV concentrations were determined in sterile filtered supernatant samples, collected during the characterization of the growth properties. Several mutants were tested. The mutant B213-PagL-KI expresses the PagL gene. PagL is a lipid A modifying enzyme and deacylates lipid A. The mutant B213/B2329-KO has a knockout of the BP2329 glycosyltransferase, resulting in a shortened oligosaccharide. B213/OmpA-D124N showed a significant increase in sOMV concentration compared to B213 (Wt) p<0.01 (FIG. 4). Both B213/PagL-KI and B213/BP2329-KO were observed to have a reduced sOMV secretion compared to B213 (Wt) with p<0.01 and p<0.001 respectively.

3.3 Characterization of sOMV Secreting Properties of B. pertussis and Mutants Based on Protein Content.

Besides the characterization of the sOMV secretion based on lipid determination by FM4-64, a second method was applied. A BCA assay was performed to quantify the protein content in isolated sOMV preparations obtained from bacterial cultures of the newly created B. pertussis mutants (B1917/BP2019-D50N and B1917/OmpA-D124N/BP2019-D50N) and in advanced created B213 LPS mutants (B213/PagL-KI and B213/BP2329-KO) with respect to the corresponding wild-type and OmpA-D124N mutant. Same isolated sOMV preparations were used as previously used during the FM4-64 assay in order to compare the results.

3.3.1. Protein Content of Purified sOMV Secreted by B1917/BP2019-D50N and B1917/OmpA-D124N/BP2019-D50N in Comparison to the B1917 (Wt) and B1917/OmpA-D124N

The same trend in protein content was observed, as previously was observed for sOMV concentration based on lipid content, determined by FM4-64 assay (FIG. 2). The protein content of the isolated sOMV preparation of B1917/OmpA-D124N/BP2019-D50N (439.16 μg/L±21.28) and increased protein content compared to the corresponding B1917 (Wt) (217.07 μg/L±27.6) with P<0.0001. A massive significant increase was observed for B1917/OmpA-D124N (826.56 μg/L±15.45) compared to B1917 (Wt), with P<0.0001, a 6.4 fold increase.

3.3.2 Protein Content of Purified sOMV Secreted by B213/PagL-KI and B213/Bp2329-KO in Comparison to the B213 (Wt) and B213/OmpA-D124N

Even as the B. pertussis B1917 strains, the same trend in protein content in isolated sOMV preparations was observed as previously shown by FM4-64 (FIG. 4 and FIG. 5). A significant increase in protein content was observed for the B213/OmpA-D124N (181.97 μg/L±6.7) compared to the B213 (Wt) (155.99 μg/L±4.59). A reduced protein content was observed for both B213/PagL-KI (120.51 μg/L±5.48) and B213/Bp2329-KO (80.83 μg/L±4.55) compared to the B213 (Wt), this reduction in protein content was significantly lower with P<0.01 and P<0.001 respectively.

3.4 Characterization of LPS Content of sOMV Secreted by B. pertussis and Mutants Based on Total Carbohydrate Concentration

LPS is known as a natural adjuvant which can enhance the immune response by stimulation of T cells producing IFN-γ and TNF. LPS concentration was determined to obtain a better view on the toxicity of the sOMV secreted by the newly created B. pertussis mutants and previously created B213 LPS mutants (B213/PagL-KI and B213/BP2329-KO). Both B213/PagL-KI and B213/BP2329-KO contain a LPS modifying mutation which could lead to an increased sOMV production by accumulation of LPS related structures in the periplasm, resulting in an increased LPS concentration in the secreted sOMV. The LPS concentration of sOMVs was determined by the phenol sulfuric acid method. Same isolated sOMV preparations were used for the LPS determination as previously used for the FM4-64 and BCA.

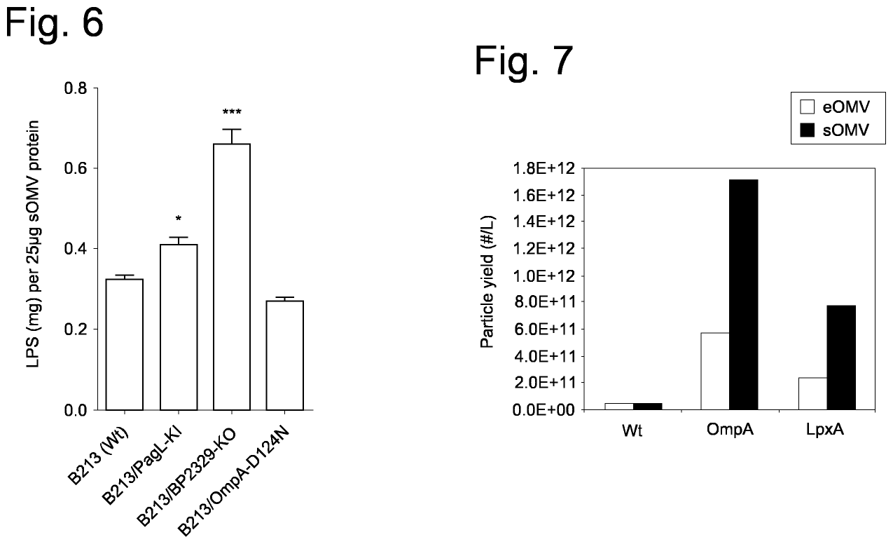

To be able to make a reliable comparison between the LPS concentration of the sOMV, secreted by B. pertussis and mutants, the LPS concentration was normalized to 25 μg protein. These protein concentrations were previously determined by a BCA. An increased concentration of LPS was observed for the B213 strains compared to the B1917 strains. A significantly increased LPS concentration was shown for B213/BP2329-KO (0.66 mg/25 μg protein±0.02) and B213/PagL-KI (0.41 mg/25 μg protein±0.01) compared to B213 (Wt) (0.325 mg/25 μg protein±0.005). For B213/OmpA-D124N (0.272 mg/25 μg protein±0.005), no significant increase was observed (FIG. 6). For the B1917 mutants B1917/OmpA-D124N and B1917/OmpA-D124N/BP2019-D50N no significant difference was shown compared to the B1917 (Wt) in sOMV derived from isolation processes (data not shown).

3.5. Increased sOMV and eOMV Production in OmpA Mutants as Well as in LpxA Mutants.

Heterologous LpxA acyltransferase activity was introduced into Bordetella pertussis to reduce LPS endotoxicity. Surprisingly, we observed that the introduction of heterologous acyltransferase activity increased OMV production (FIG. 7). As compared to wild type, the OMV production of sOMVs as well as eOMVs was significantly increased after heterologous LpxA expression.

3.6 Increased of Protective Immunity after Retaining of Prn to the Outer Membrane of the OMV.

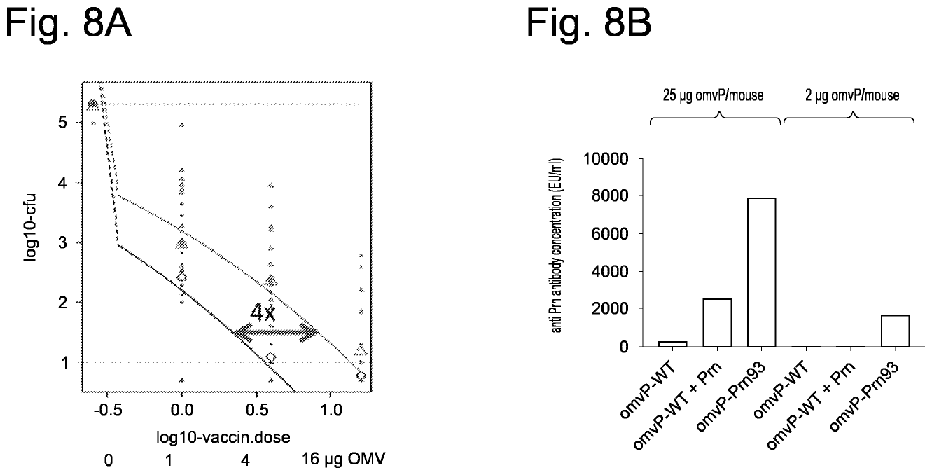

Enhanced immunogenicity of membrane bound prn93 was demonstrated by comparing OMV-prn93 and a mixture of OMV-WT and purified prn69 (FIG. 8). OMV-prn93 contains approximately 3 μg Pm per human dose (50 μg), similar to acP (acellular pertussis vaccine). Comparison of specific anti-Prn antibody responses showed that OMV-Prn93 induced 10 times higher anti-Prn antibody titers compared to 3 μg purified Prn69 mixed with OMV-WT. The protection against intranasal challenge after vaccination of mice with OMV-WT or OMV-Prn is 4 times better with OMV-Prn.

1. A polypeptide comprising a sequence having at least 50% sequence identity with SEQ ID NO: 1 and comprising a mutation in an OmpA-like domain, wherein preferably the mutation is located at a position corresponding to any one of positions 110-140 in SEQ ID NO: 1, and wherein the polypeptide comprising the mutation increases OMV production when expressed in Bordetella as compared to an otherwise identical polypeptide not comprising said mutation.

2. A polypeptide according to claim 1, wherein the mutation is a mutation of a single amino acid residue.

3. A polypeptide according to claim 1, wherein the mutation is a substitution of an amino acid residue.

4. (canceled)

5. A Bordetella bacterium comprising a genomic modification in a gene encoding a polypeptide having at least 50% sequence identity with SEQ ID NO: 1.

6. A Bordetella bacterium comprising a genomic modification in a gene encoding a polpeptide having at least 50% sequence identity with SEQ ID NO: 1, wherein the genomic modification results in the expression of a polypeptide as defined in claim 1 and/or wherein the genomic modification is in a gene comprising a sequence having at least 50% sequence identity with SEQ ID NO: 2.

7. A Bordetella bacterium according to claim 5, wherein the Bordetella bacterium is at least one of B. pertussis, B. parapertussis and B. bronchiseptica.

8. A Bordetella bacterium according to claim 5, wherein the bacterium further comprises a mutation in at least one of:
i) an endogenous gene encoding LpxA;
ii) an endogenous gene encoding Pertactin.

9. A Bordetella bacterium according to a wherein the bacterium further comprises a mutation in at least one of:
i) an endogenous gene encoding Ptx; and
ii) an endogenous gene encoding DNT.

10. (canceled)

11. (canceled)

12. A composition comprising at least one of:
i) a Bordetella bacterium comprising a genomic SEQ ID NO: 1, wherein preferably the bacterium is an inactivated bacterium; and
ii) an OMV obtainable from a Bordetella bacterium comprising a genomic modification in a gene encoding a polypeptide having at least 50% sequence identity with SEQ ID NO: 1.

13.-15. (canceled)

16. The polypeptide according to claim 3, wherein the substitution is at a position corresponding to position 124 of SEQ ID NO: 1.

17. The polypeptide according to claim 16, wherein the substitution is a D124N substitution.

18. A Bordetella bacterium according to claim 5, wherein the modification is located in the open reading frame of the gene.

19. A Bordetella bacterium according to claim 5, wherein the modification increases OMV (Outer Membrane Vesicle) production of the Bordetella bacterium as compared to the same bacterium not comprising the modification.

20. The composition according to claim 12, wherein the composition is a pharmaceutical composition.


SEQUENCE LISTING (18 sequences)

SEQ ID NO: 1 - protein, 193 aa, Bordetella pertussis
MNKPSKFALALAFAAVTASGAASAQTVDNWRNPFGDVWKNGTNELCWRDAFWTPATGIPGCDGVPVAQKEKSAPMAAKVVFNADTFFDFDKSTLKPEGRQLLDQVAQQAGTIDLETIIAVGHTDSIGTEAYNQKLSERRAAAVKTYLVSKGIDPNRIYTEGKGELQPIASNKTREGRAQNRRVEIEIVGSRKN

SEQ ID NO: 2 - DNA, 582 bp, Bordetella pertussis
atgaacaaaccctccaaattcgctctggcgctcgccttcgccgctgttacggcctctggtgcagcttccgcgcaaaccgtggacaactggcgcaatccgtttggcgacgtttggaagaacggcaccaatgaactgtgctggcgcgatgcgttctggaccccggctaccggcatccccggttgcgacggcgttccggtcgctcagaaggaaaagtccgctcccatggccgccaaggtcgtgttcaatgctgacaccttcttcgacttcgacaagtcgacgctgaagccggaaggccgccagctgctggatcaagtcgcccagcaagccggcacgatcgatctggaaacgatcatcgccgttggccacacggactcgatcggcaccgaagcctacaaccagaagctgtccgagcgccgtgccgctgcggtcaagacctacctggtcagcaagggtatcgaccccaaccgtatctacacggaaggcaagggcgaactgcaaccgatcgcttcgaacaagacgcgtgaaggccgtgcccagaaccgtcgcgtggaaatcgaaatcgtcggtagccgcaagaactaa

SEQ ID NO: 3 - protein, 193 aa, artificial sequence
  OmpA D124N
MNKPSKFALALAFAAVTASGAASAQTVDNWRNPFGDVWKNGTNELCWRDAFWTPATGIPGCDGVPVAQKEKSAPMAAKVVFNADTFFDFDKSTLKPEGRQLLDQVAQQAGTIDLETIIAVGHTNSIGTEAYNQKLSERRAAAVKTYLVSKGIDPNRIYTEGKGELQPIASNKTREGRAQNRRVEIEIVGSRKN

SEQ ID NO: 4 - DNA, 582 bp, artificial sequence
  OmpA D124N
atgaacaaaccctccaaattcgctctggcgctcgccttcgccgctgttacggcctctggtgcagcttccgcgcaaaccgtggacaactggcgcaatccgtttggcgacgtttggaagaacggcaccaatgaactgtgctggcgcgatgcgttctggaccccggctaccggcatccccggttgcgacggcgttccggtcgctcagaaggaaaagtccgctcccatggccgccaaggtcgtgttcaatgctgacaccttcttcgacttcgacaagtcgacgctgaagccggaaggccgccagctgctggatcaagtcgcccagcaagccggcacgatcgatctggaaacgatcatcgccgttggccacacgaactcgatcggcaccgaagcctacaaccagaagctgtccgagcgccgtgccgctgcggtcaagacctacctggtcagcaagggtatcgaccccaaccgtatctacacggaaggcaagggcgaactgcaaccgatcgcttcgaacaagacgcgtgaaggccgtgcccagaaccgtcgcgtggaaatcgaaatcgtcggtagccgcaagaactaa

SEQ ID NO: 5 - protein, 915 aa, Bordetella pertussis
MNMSLSRIVKAAPLRRTTLAMALGALGAAPAAHADWNNQSIVKTGERQHGIHIQGSDPGGVRTASGTTIKVSGRQAQGILLENPAAELQFRNGSVTSSGQLSDDGIRRFLGTVTVKAGKLVADHATLANVGDTWDDDGIALYVAGEQAQASIADSTLQGAGGVQIERGANVTVQRSAIVDGGLHIGALQSLQPEDLPPSRVVLRDTNVTAVPASGAPAAVSVLGASELTLDGGHITGGRAAGVAAMQGAVVHLQRATIRRGDAPAGGAVPGGAVPGGFGPGGFGPGGFGPGGFGPVLDGWYGVDVSGSSVELAQSIVEAPELGAAIRVGRGARVTVSGGSLSAPHGNVIETGGARRFAPQAAPLSITLQAGAHAQGKALLYRVLPEPVKLTLTGGADAQGDIVATELPSIPGTSIGPLDVALASQARWTGATRAVDSLSIDNATWVMTDNSNVGALRLASDGSVDFQQPAEAGRFKVLTVNTLAGSGLFRMNVFADLGLSDKLVVMQDASGQHRLWVRNSGSEPASANTLLLVQTPLGSAATFTLANKDGKVDIGTYRYRLAANGNGQWSLVGAKAPPAPKPAPQPGPQPPQPPQPQPEAPAPQPPAGRELSAAANAAVNTGGVGLASTLWYAESNALSKRLGELRLNPDAGGAWGRGFAQRQQLDNRAGRRFDQKVAGFELGADHAVAVAGGRWHLGGLAGYTRGDRGFTGDGGGHTDSVHVGGYATYIADSGFYLDATLRASRLENDFKVAGSDGYAVKGKYRTHGVGASLEAGRRFTHADGWFLEPQAELAVFRAGGGAYRAANGLRVRDEGGSSVLGRLGLEVGKRIELAGGRQVQPYIKASVLQEFDGAGTVHTNGIAHRTELRGTRAELGLGMAAALGRGHSLYASYEYSKGPKLAMPWTFHAGYRYSW

SEQ ID NO: 6 - protein, 258 aa, Pseudomonas aeruginosa
MSLIDPRAIIDPSARLAADVQVGPWSIVGAEVEIGEGTVIGPHVVLKGPTKIGKHNRIYQFSSVGEDTPDLKYKGEPTRLVIGDHNVIREGVTIHRGTVQDRAETTIGDHNLIMAYAHIGHDSVIGNHCILVNNTALAGHVHVDDWAILSGYTLVHQYCRIGAHSFSGMGSAIGKDVPAYVTVFGNPAEARSMNFEGMRRRGFSSEAIHALRRAYKVVYRQGHTVEEALAELAESAAQFPEVAVFRDSIQSATRGITR

SEQ ID NO: 7 - protein, 353 aa, Pseudomonas aeruginosa
MMSTLSYTLGQLAAHVGAELRGDADLPIQGLATLQEAGPAQLSFLANPQYRKYLPESRAGAVLLTAADANGFSGTALVVANPYLAYASLSHLFDRKPKAAAGIHPTAIVAADAEVDPSASVGAYAVIESGARIGAGVSIGAHCVIGARSVIGEGGWLAPRVTLYHDVTIGARVSIQSGAVIGGEGFGFANEKGVWQKIAQIGGVTIGDDVEIGANTTIDRGALSDTLIGNGVKLDNQIMIAHNVQIGDHTAMAACVGISGSAKIGRHCMLAGGVGLVGHIEICDNVFVTGMTMVTRSITEPGSYSSGTAMQPAAEWKKSAARIRQLDDMARRLQQLEKRLAAVTSSGDASSDA

SEQ ID NO: 8 - protein, 193 aa, Bordetella parapertussis
MNKPSKFALALAFAAVTASGAASAQTVDNWRNPFGDVWKNGTNELCWRDAFWTPATGIPGCDGVPVAQKEKPAPMAAKVVFNADTFFDFDKSTLKPEGRQLLDQVAQQAGTIDLETIIAVGHTDSIGTEAYNQKLSERRAAAVKTYLVSKGIDPNRIYTEGKGELQPIASNKTREGRAQNRRVEIEIVGSRKN

SEQ ID NO: 9 - protein, 193 aa, Bordetella bronchiseptica; identical to SEQ ID NO: 8

SEQ ID NO: 10 - protein, 1464 aa, Bordetella pertussis
MDKDESALRQLVDMALVGYDGVVEELLALPSEESGDLAGGRAKREKAEFALFSEAPNGDEPIGQDARTWFYFPKYRPVAVSNLKKMQVAIRARLEPESLILQWLIALDVYLGVLIAALSRTVISDLVFEYVKARYEIYYLLNRVPHPLATAYLKRRRQRPVDRSGRLGSVFEHPLWFAYDELAGTVDLDADIYEQALAESIERRMDGEPDDGSLDTAEHDVWRLCRDGINRGEQAIFQASGPYGVVADAGYMRTVADLAYADALADCLHAQLRIRAQGSVDSPGDEMPRKLDAWEIAKFHLAATQQARVDLLEAAFALDYAALRDVRVYGDYRNALALRFIKREALRLLGARRGNASTMPAVAAGEYDEIVASGAANDAAYVSMAAALIAGVLCDLESAQRTLPVVLARFRPLGVLARFRRLEQETAGMLLGDQEPEPRGFISFTDFRDSDAFASYAEYAAQFNDYIDQYSILEAQRLARILALGSRMTVDQWCLPLQKVRHYKVLTSQPGLIARGIENHNRGIEYCLGRPPLTDLPGLFTMFQLHDSSWLLVSNINGELWSDVLANAEVMQNPTLAALAEPQGRFRTGRRTGGWFLGGPATEGPSLRDNYLLKLRQSNPGLDVKKCWYFGYRQEYRLPAGALGVPLFAVSVALRHSLDDLAAHAKSALYKPSEWQKFAFWIVPFYREIFFSTQDRSYRVDVGSIVFDSISLLASVFSIGGKLGSFTRTQYGNLRNFVVRQRIAGLSGQRLWRSVLKELPALIGASGLRLSRSLLVDLYEIFEPVPIRRLVAGFVSATTVGGRNQAFLRQAFSAASSSAGRTGGQLASEWRMAGVDATGLVESTSGGRFEGIYTRGLGPLSECTEHFIVESGNAYRVIWDAYTHGWRVVNGRLPPRLTYTVPVRLNGQGHWETHLDVPGRGGAPEIFGRIRTRNLVALAAEQAAPMRRLLNQARRVALRHIDTCRSRLALPRAESDMDAAIRIFFGEPDAGLRQRIGRRLQEVRAYIGDLSPVNDVLYRAGYDLDDVATLFNAVDRNTSLGRQARMELYLDAIVDLHARLGYENARFVDLMAFHLLSLGHAATASEVVEAVSPRLLGNVFDISNVAQLERGIGNPASTGLFVMLGAYSESSPAIFQSFVNDIFPAWRQASGGGPLVWNFGPAAISPTRLDYANTDIGLLNHGDISPLRARPPLGGRRDIDLPPGLDISFVRYDRPVRMSAPRALDASVFRPVDGPVHGYIQSWTGAEIEYAYGAPAAAREVMLTDNVRIISIENGDEGAIGVRVRLDTVPVATPLILTGGSLSGCTTMVGVKEGYLAFYHTGKSTELGDWATAREGVQALYQAHLAMGYAPISIPAPMRNDDLVSIAATYDRAVIAYLGKDVPGGGSTRITRHDEGAGSVVSFDYNAAVQASAVPRLGQVYVLISNDGQGARAVLLAEDLAWAGSGSALDVLNERLVTLFPAPV

SEQ ID NO: 11 - protein, 915 aa, artificial sequence
  Pertactin D738N mutation
MNMSLSRIVKAAPLRRTTLAMALGALGAAPAAHADWNNQSIVKTGERQHGIHIQGSDPGGVRTASGTTIKVSGRQAQGILLENPAAELQFRNGSVTSSGQLSDDGIRRFLGTVTVKAGKLVADHATLANVGDTWDDDGIALYVAGEQAQASIADSTLQGAGGVQIERGANVTVQRSAIVDGGLHIGALQSLQPEDLPPSRVVLRDTNVTAVPASGAPAAVSVLGASELTLDGGHITGGRAAGVAAMQGAVVHLQRATIRRGDAPAGGAVPGGAVPGGFGPGGFGPGGFGPGGFGPVLDGWYGVDVSGSSVELAQSIVEAPELGAAIRVGRGARVTVSGGSLSAPHGNVIETGGARRFAPQAAPLSITLQAGAHAQGKALLYRVLPEPVKLTLTGGADAQGDIVATELPSIPGTSIGPLDVALASQARWTGATRAVDSLSIDNATWVMTDNSNVGALRLASDGSVDFQQPAEAGRFKVLTVNTLAGSGLFRMNVFADLGLSDKLVVMQDASGQHRLWVRNSGSEPASANTLLLVQTPLGSAATFTLANKDGKVDIGTYRYRLAANGNGQWSLVGAKAPPAPKPAPQPGPQPPQPPQPQPEAPAPQPPAGRELSAAANAAVNTGGVGLASTLWYAESNALSKRLGELRLNPDAGGAWGRGFAQRQQLDNRAGRRFDQKVAGFELGADHAVAVAGGRWHLGGLAGYTRGDRGFTGDGGGHTDSVHVGGYATYIADSGFYLNATLRASRLENDFKVAGSDGYAVKGKYRTHGVGASLEAGRRFTHADGWFLEPQAELAVFRAGGGAYRAANGLRVRDEGGSSVLGRLGLEVGKRIELAGGRQVQPYIKASVLQEFDGAGTVHTNGIAHRTELRGTRAELGLGMAAALGRGHSLYASYEYSKGPKLAMPWTFHAGYRYSW

SEQ ID NO: 12 - protein, 269 aa, Bordetella pertussis
MRCTRAIRQTARTGWLTWLAILAVTAPVTSPAWADDPPATVYRYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSEYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 13 - DNA, 20 bp, artificial sequence
  primer B1917-OmpA-Fw
ggtcaatgcaacggtctagg

SEQ ID NO: 14 - DNA, 24 bp, artificial sequence
  primer OmpA-D124N-Rv
gccgatcgagttcgtgtggccaac

SEQ ID NO: 15 - DNA, 22 bp, artificial sequence
  primer OmpA-D124N-Fw
ttggccacacgaactcgatcgg

SEQ ID NO: 16 - DNA, 18 bp, artificial sequence
  primer B1917-OmpA-Rv
atgctctccgacaggatg

SEQ ID NO: 17 - DNA, 18 bp, artificial sequence
  primer OmpA-seq-Fw
cgtatgtaaggatgaacc

SEQ ID NO: 18 - DNA, 18 bp, artificial sequence
  primer OmpA-seq-Rv2
tgttcgagcatttccatg